(19) United States
(12) Patent Application Publication
(10) Pub. No.: US 2023/0255558 A1
(43) Pub. Date: Aug. 17, 2023

(54) HEAD TILT MOTION ASSISTANCE SYSTEM AND HEAD TILT MOTION ASSISTANCE PROGRAM

(71) Applicant: Katsunori Suetsugu, Tokyo (JP)

(72) Inventor: Katsunori Suetsugu, Tokyo (JP)

(21) Appl. No.: 18/109,071

(22) Filed: Feb. 13, 2023

(30) Foreign Application Priority Data
Feb. 17, 2022 (JP) 2022-023178
Oct. 11, 2022 (JP) 2022-163122

(51) Int. Cl. A61B 5/00

(52) U.S. Cl. CPC A61B 5/6803; A61M 2021/0027

(57) ABSTRACT

A head tilt motion assistance system includes a head tilt detection unit that detects a tilt of a head of a user, and a motion assistance notification unit that makes a motion assistance notification that is a notification for assisting in a head tilt motion that is a motion in which the user tilts the head, and the motion assistance notification unit makes the motion assistance notification on the basis of the tilt detected by the head tilt detection unit.

### CROSS REFERENCE TO RELATED APPLICATION

This application is based upon and claims the benefit of priority from Japanese Patent Application No. 2022-023178 filed Feb. 17, 2022 and Japanese Patent Application No. 2022-163122 filed Oct. 11, 2022, the entire contents of which are incorporated herein by reference.

### BACKGROUND OF THE INVENTION

### 1. Technical Field

The present invention relates to a head tilt motion assistance system and a head tilt motion assistance program that assist a user in tilting his/her head.

### 2. Description of the Related Art

When stuff that makes a brain fatigued (hereinafter, referred to as “brain fatigue stuff”) such as amyloid β accumulates in the brain, the brain gets fatigued. The brain fatigue stuff accumulates in the brain not only in a case where a human is actively using the brain but also in a case where the human is only awake without sleeping.

It is known that cerebrospinal fluid contributes to removal of the brain fatigue stuff from the brain, and a flow rate of the cerebrospinal fluid in the brain increases during sleep or under anesthesia to increase a removal rate of the brain fatigue stuff. A brain function improving agent for increasing efficiency of the removal of the brain fatigue stuff from the brain using the cerebrospinal fluid is also known (see, for example, JP 2018-052913 A).

### SUMMARY OF THE INVENTION

The technique in the related art, however, requires that the brain function improving agent be administered to a user by oral administration, intravenous administration, or the like.

It is therefore an object of the present invention to provide a head tilt motion assistance system and a head tilt motion assistance program capable of assisting a user in recovering from brain fatigue even with no brain function improving agent administered to the user.

A head tilt motion assistance system according to the present invention includes a head tilt detection unit that detects a tilt of a head of a user, and a motion assistance notification unit that makes a motion assistance notification that is a notification for assisting in a head tilt motion that is a motion in which the user tilts the head, and the motion assistance notification unit makes the motion assistance notification on the basis of the tilt detected by the head tilt detection unit.

This configuration causes the head tilt motion assistance system according to the present invention to make the motion assistance notification on the basis of the tilt of the head of the user detected by the head tilt detection unit, so that it is possible to increase the possibility that the head tilt motion that is highly likely to cause the user to effectively recover from the fatigue of the brain is appropriately executed by the user. This allows the head tilt motion assistance system according to the present invention to assist the user in recovering from the fatigue of the brain even with no brain function improving agent administered to the user.

In the head tilt motion assistance system according to the present invention, the motion assistance notification may include a notification of a score of the head tilt motion, the motion assistance notification unit may identify an actual execution detail of the head tilt motion on a basis of the tilt detected by the head tilt detection unit, the motion assistance notification unit may determine the score on a basis of on at least score determination criteria that are criteria for determination of the score and the actual execution detail of the head tilt motion, and the score determination criteria may include a criterion according to an execution detail of the head tilt motion.

This configuration causes the head tilt motion assistance system according to the present invention to determine the score of the head tilt motion on the basis of at least the score determination criteria including a criterion according to the execution detail of the head tilt motion and the actual execution detail of the head tilt motion, and notifies the determined score, so that it is possible to increase the possibility that the head tilt motion is appropriately executed by the user.

In the head tilt motion assistance system according to the present invention, the score determination criteria may include a criterion for increasing the score when, in a case where a stop of tilt change that is a stop of change in the tilt in the head tilt motion is executed in a state where the tilt exists in a first area among a plurality of areas obtained by dividing a space into the plurality of areas with a center point of the tilt as a center, the stop of tilt change executed immediately after the stop of tilt change executed in a state where the tilt exists in the first area is the stop of tilt change executed in a state where the tilt exists in a second area among the plurality of areas, and the second area exists in a direction opposite to the first area.

This configuration causes, when, in a case where the stop of tilt change is executed in a state where the tilt of the head of the user exists in the first area, the stop of tilt change executed immediately after the stop of tilt change executed in a state where the tilt exists in the first area is the stop of tilt change executed in a state where the tilt of the head of the user exists in the second area existing in the direction opposite to the first area, the head tilt motion assistance system according to the present invention to increase the score of the head tilt motion, so that it is possible to increase the possibility that the head tilt motion that causes the cerebrospinal fluid to efficiently flow in the brain is executed by the user.

In the head tilt motion assistance system according to the present invention, the score determination criteria may include a criterion for increasing the score in a case where a stop of change in the tilt in the head tilt motion is executed in all a state where the tilt exists in a first area among a plurality of areas obtained by dividing a space into the plurality of areas with a center point of the tilt as a center, a state where the tilt exists in a second area among the plurality of areas, a state where the tilt exists in a third area among the plurality of areas, and a state where the tilt exists in a fourth area among the plurality of areas, the second area exists in a direction opposite to the first area, the third area exists in a direction orthogonal to the second area, and the fourth area exists in a direction opposite to the third area.

This configuration causes the head tilt motion assistance system according to the present invention to increase the score of the head tilt motion in a case where the stop of tilt change of the head of the user is executed in the head tilt motion in all of the state where the tilt of the head of the user exists in the first area, the state where the tilt of the head of the user exists in the second area existing in the direction opposite to the first area, the state where the tilt of the head of the user exists in the third area existing in the direction orthogonal to the second area, and the state where the tilt of the head of the user exists in the fourth area existing in the direction opposite to the third area, so that it is possible to increase the possibility that the head tilt motion that causes the cerebrospinal fluid to efficiently flow in the brain is executed by the user.

In the head tilt motion assistance system according to the present invention, the score determination criteria may include a criterion according to duration of a stop of change in the tilt in the head tilt motion, and the motion assistance notification unit may determine the score on a basis of at least the score determination criteria and actual duration of the stop of change in the tilt in the head tilt motion.

This configuration causes the head tilt motion assistance system according to the present invention to determine the score of the head tilt motion on the basis of at least the score determination criteria including the criterion according to the duration of the stop of tilt change of the head of the user in the head tilt motion and the actual duration of the stop of tilt change of the head of the user in the head tilt motion, so that it is possible to increase the possibility that the head tilt motion that causes the cerebrospinal fluid to efficiently flow in the brain is executed by the user.

In the head tilt motion assistance system according to the present invention, the score determination criteria may include a criterion according to a relaxation degree of the user in the head tilt motion, and the motion assistance notification unit may determine the score on a basis of at least the score determination criteria and an actual relaxation degree of the user in the head tilt motion.

This configuration causes the head tilt motion assistance system according to the present invention to determine the score of the head tilt motion on the basis of at least the score determination criteria including the criterion according to the relaxation degree of the user in the head tilt motion and the actual relaxation degree of the user in the head tilt motion, so that it is possible to increase the possibility that the head tilt motion is executed by the user in a state where the relaxation degree is high, that is, in a state where the user easily recovers from the fatigue of the brain.

In the head tilt motion assistance system according to the present invention, the score determination criteria may include a criterion according to an execution detail of preprocessing of the head tilt motion, and the motion assistance notification unit may determine the score on a basis of at least the score determination criteria and an actual execution detail of the preprocessing of the head tilt motion.

This configuration causes the head tilt motion assistance system according to the present invention to determine the score of the head tilt motion on the basis of at least the score determination criteria including the criterion according to the execution detail of the preprocessing of the head tilt motion and the actual execution detail of the preprocessing of the head tilt motion, so that it is possible to increase the possibility that the preprocessing that causes the cerebrospinal fluid to efficiently flow in the brain is executed by the user.

In the head tilt motion assistance system according to the present invention, the motion assistance notification may include a notification of a reason for the score.

This configuration causes the head tilt motion assistance system according to the present invention to make a notification of the reason for the score of the head tilt motion, so that it is possible to increase the possibility that the head tilt motion is appropriately executed by the user.

In the head tilt motion assistance system according to the present invention, the motion assistance notification unit may set the score determination criteria for a case where a fatigue degree of a brain of the user after execution of the head tilt motion is greater than or equal to a specific degree such that the score is determined to be low as compared with the score determination criteria for a case where the fatigue degree of the brain of the user after execution of the head tilt motion is less than the specific degree.

This configuration causes the head tilt motion assistance system according to the present invention to set the score determination criteria such that the score of the head tilt motion is determined to be low in a case where the brain fatigue degree of the user is low after the execution of the head tilt motion, so that it is possible to increase the possibility that the head tilt motion that becomes high in score in a case where the brain fatigue degree of the user is low after the execution of the head tilt motion is positively executed by the user. This allows the head tilt motion assistance system according to the present invention to increase the possibility that the head tilt motion is appropriately executed by the user.

In the head tilt motion assistance system according to the present invention, the motion assistance notification may include a notification of a state of a stop of change in the tilt, and the motion assistance notification unit may make the notification of the state of the stop of change in the tilt during execution of the head tilt motion.

This configuration causes the head tilt motion assistance system according to the present invention to make a notification of the state of the stop of tilt change of the head of the user during the execution of the head tilt motion, so that it is possible to increase the possibility that the head tilt motion that causes the cerebrospinal fluid to efficiently flow in the brain is executed by the user.

In the head tilt motion assistance system according to the present invention, the motion assistance notification may include a notification of a relaxation degree of the user, and the motion assistance notification unit may make the notification of the relaxation degree of the user during execution of the head tilt motion.

This configuration causes the head tilt motion assistance system according to the present invention to make a notification of the relaxation degree of the user during the execution of the head tilt motion, so that it is possible to cause the user to aim for a state where the relaxation degree is high, that is, a state where the user easily recovers from the fatigue of the brain during the execution of the head tilt motion.

In the head tilt motion assistance system according to the present invention, the motion assistance notification may include a notification of an instruction to execute preprocessing of the head tilt motion, and the motion assistance notification unit may make the notification of the instruction to execute the preprocessing of the head tilt motion before executing of the head tilt motion.

This configuration causes the head tilt motion assistance system according to the present invention to make a notification of the instruction to execute the preprocessing of the head tilt motion before executing the head tilt motion, so that it is possible to increase the possibility that the preprocessing that causes the cerebrospinal fluid to efficiently flow in the brain is executed by the user.

In the head tilt motion assistance system according to the present invention, the motion assistance notification may include a notification of the tilt that is a recommended tilt, the motion assistance notification unit may make the notification of the recommended tilt during execution of the head tilt motion, the motion assistance notification unit may identify an actual execution detail of the head tilt motion on a basis of the tilt detected by the head tilt detection unit, the motion assistance notification unit may determine the recommended tilt on a basis of at least the actual execution detail of the head tilt motion and recommended tilt determination criteria that are criterions for determination of the recommended tilt, and the recommended tilt determination criteria may include a criterion according to an execution detail of the head tilt motion.

This configuration causes the head tilt motion assistance system according to the present invention to determine the recommended tilt of the head of the user on the basis of at least the recommended tilt determination criteria including the criterion according to the execution detail of the head tilt motion and the actual execution detail of the head tilt motion, and makes a notification of the recommended tilt of the head of the user during the execution of the head tilt motion, so that it is possible to increase the possibility that the head tilt motion that causes the user to effectively recover from the fatigue of the brain is appropriately executed by the user.

In the head tilt motion assistance system according to the present invention, the recommended tilt determination criteria may include a criterion for determining the tilt in a second area among a plurality of areas obtained by dividing a space into the plurality of areas with a center point of the tilt as a center as the recommended tilt when, in a case where a stop of tilt change that is a stop of change in the tilt in the head tilt motion is executed in a state where the tilt exists in a first area among the plurality of areas, the stop of tilt change executed immediately before the stop of tilt change executed in a state where the tilt exists in the first area is not the stop of tilt change executed in a state where the tilt exists in the second area, and the second area exists in a direction opposite to the first area.

This configuration causes the head tilt motion assistance system according to the present invention to determine the tilt in the second area existing in the direction opposite to the first area as the recommended tilt of the head of the user when, in a case where the stop of tilt change is executed in a state where the tilt of the head of the user exists in the first area, the stop of tilt change executed immediately before the stop of tilt change executed in a state where the tilt exists in the first area is not the stop of tilt change executed in a state where the tilt of the head of the user exists in the second area, so that it is possible to increase the possibility that the head tilt motion that causes the cerebrospinal fluid to efficiently flow in the brain is executed by the user.

In the head tilt motion assistance system according to the present invention, the recommended tilt determination criteria may include a criterion for determining the tilt in a third area among the plurality of areas as the recommended tilt when, in a case where the stop of tilt change is executed in a state where the tilt exists in the first area, the stop of tilt change executed immediately before the stop of tilt change executed in a state where the tilt exists in the first area is the stop of tilt change executed in a state where the tilt exists in the second area, and the third area exists in a direction orthogonal to the second area.

This configuration causes the head tilt motion assistance system according to the present invention to determine the tilt in the third area existing in the direction orthogonal to the second area as the recommended tilt of the head of the user when, in a case where the stop of tilt change is executed in a state where the tilt of the head of the user exists in the first area, the stop of tilt change executed immediately before the stop of tilt change executed in a state where the tilt exists in the first area is the stop of tilt change executed in a state where the tilt of the head of the user exists in the second area existing in the direction opposite to the first area, so that it is possible to increase the possibility that the head tilt motion that causes the cerebrospinal fluid to efficiently flow in the brain is executed by the user.

The head tilt motion assistance system according to the present invention may further include a γ wave light output unit that outputs γ wave light that is light blinking at a frequency identical to a frequency of a γ wave of brain waves, and the motion assistance notification unit may cause the γ wave light output unit to output the γ wave light in the head tilt motion.

This configuration causes the head tilt motion assistance system according to the present invention to cause the γ wave light output unit to output the γ wave light in the head tilt motion. Therefore, the γ wave light that is output from the γ wave light output unit comes within sight of the user during the execution of the head tilt motion, so that it is possible to effectively remove the brain fatigue stuff from the brain of the user.

The head tilt motion assistance system according to the present invention may further include a γ wave sound output unit that outputs γ wave sound that is sound identical in frequency to a γ wave of brain waves, and the motion assistance notification unit may cause the γ wave sound output unit to output the γ wave sound in the head tilt motion.

This configuration causes the head tilt motion assistance system according to the present invention to cause the γ wave sound output unit to output the γ wave sound in the head tilt motion. Therefore, the γ wave sound that is output from the γ wave sound output unit comes within hearing of the user during the execution of the head tilt motion, so that it is possible to effectively remove the brain fatigue stuff from the brain of the user.

In the head tilt motion assistance system according to the present invention, the motion assistance notification unit may execute a test for measuring a fatigue degree of a brain of the user after execution of the head tilt motion, and the motion assistance notification may include a notification of the fatigue degree of the brain measured by the test.

This configuration causes the head tilt motion assistance system according to the present invention to execute the test for measuring the fatigue degree of the brain of the user after execution of the head tilt motion and make a notification of the fatigue degree of the brain measured by the test, so that it is possible for the user to grasp the effect of the head tilt motion. This allows the head tilt motion assistance system according to the present invention to cause the user to aim to execute a head tilt motion that causes the user to effectively recover from the fatigue of the brain.

In the head tilt motion assistance system according to the present invention, the motion assistance notification may include a notification of an image representing a state where cerebrospinal fluid moves in a brain in a gravity direction, and the motion assistance notification unit may identify a gravity direction with respect to the head on a basis of the tilt detected by the head tilt detection unit in the head tilt motion, and change the gravity direction with respect to the brain in the image according to the gravity direction identified.

This configuration causes the head tilt motion assistance system according to the present invention to identify, in a case where a notification of the image representing a state where the cerebrospinal fluid moves in the brain in the gravity direction is made, the gravity direction with respect to the head of the user on the basis of the tilt of the head detected by the head tilt detection unit in the head tilt motion, and change the gravity direction with respect to the brain in the image representing a state where the cerebrospinal fluid moves in the brain in the gravity direction according to the identified gravity direction with respect to the head, so that it is possible to cause the user to aim to execute a head tilt motion that causes the cerebrospinal fluid to efficiently move in the brain in the image. This allows the head tilt motion assistance system according to the present invention to cause the user to aim to execute a head tilt motion that causes the user to effectively recover from the fatigue of the brain.

A non-transitory computer-readable storage medium storing a head tilt motion assistance program according to the present invention causes a computer to implement a motion assistance notification unit that makes a motion assistance notification that is a notification for assisting in a head tilt motion that is a motion in which a user tilts a head of the user, and the motion assistance notification unit makes the motion assistance notification on a basis of the tilt detected by a head tilt detection unit that detects the tilt of the head.

This configuration causes the computer that executes the head tilt motion assistance program according to the present invention to make the motion assistance notification on the basis of the tilt of the head of the user detected by the head tilt detection unit, so that it is possible to increase the possibility that the head tilt motion that is highly likely to cause the user to effectively recover from the fatigue of the brain is appropriately executed by the user. This allows the computer that executes the head tilt motion assistance program according to the present invention to assist the user in recovering from the fatigue of the brain even with no brain function improving agent administered to the user.

The head tilt motion assistance system and the head tilt motion assistance program according to the present invention can assist a user in recovering from the fatigue of the brain even with no brain function improving agent administered to the user.

### BRIEF DESCRIPTION OF THE DRAWINGS

FIG. 1 is a block diagram of a head tilt motion assistance system according to a first embodiment of the present invention;

FIG. 2 is a diagram showing a tilt detected by a head tilt detection unit shown in FIG. 1;

FIG. 3 is a diagram showing a list of areas that may include the tilt detected by the head tilt detection unit shown in FIG. 1;

FIG. 4 is a diagram showing an example of setting information shown in FIG. 1;

FIG. 5 is a diagram showing an example of score determination information shown in FIG. 1;

FIG. 6A is a diagram showing an example of a criterion for a score of preprocessing of a head tilt motion among the criteria included in the score determination criteria shown in FIG. 1; FIG. 6B is a diagram showing an example of a criterion for a score of output of γ wave light and γ wave sound among the criteria included in the score determination criteria shown in FIG. 1;

FIG. 7A is a diagram showing an example of a criterion for a score of a tilt and stop motion among the criteria included in the score determination criteria shown in FIG. 1; FIG. 7B is a diagram showing an example of a criterion for a reduction rate of a score of the second and subsequent stop of tilt change for the same area among the criteria included in the score determination criteria shown in FIG. 1; FIG. 7C is a diagram showing an example of a criterion for an increase rate of a score in a case where the stop of tilt change for each area in a pair of areas in opposite directions is successively executed among the criteria included in the score determination criteria shown in FIG. 1; FIG. 7D is a diagram showing an example of a criterion for an increase rate of a score in a case where the stop of tilt change is executed for each area in an orthogonal combination among the criteria included in the score determination criteria shown in FIG. 1;

FIG. 8 is a flowchart of a part of operation of the head tilt motion assistance system shown in FIG. 1 in a case where the start of the head tilt motion is instructed;

FIG. 9 is a flowchart of operation subsequent to the operation shown in FIG. 8;

FIG. 10 is a flowchart of operation subsequent to the operation shown in FIG. 9;

FIG. 11 is a flowchart of operation subsequent to the operation shown in FIG. 10;

FIG. 12 is a flowchart of initial motion processing shown in FIGS. 8 to 11;

FIG. 13 is a flowchart showing an example of score determination processing shown in FIGS. 8 to 11;

FIG. 14 is a flowchart of operation of the head tilt motion assistance system shown in FIG. 1 in a case where a cerebrospinal fluid movement image is displayed after the head tilt motion;

FIG. 15A is a diagram showing an example of the cerebrospinal fluid movement image displayed on a display unit shown in FIG. 1 at a specific timing in the head tilt motion; FIG. 15B is a diagram showing an example of the cerebrospinal fluid movement image displayed on the display unit at a specific timing after the timing shown in FIG. 15A; FIG. 15C is a diagram showing an example of the cerebrospinal fluid movement image displayed on the display unit at a specific timing after the timing shown in FIG. 15B;

FIG. 16A is a diagram showing an example of the cerebrospinal fluid movement image displayed on the display unit at a specific timing after the timing shown in FIG. 15C; FIG. 16B is a diagram showing an example of the cerebrospinal fluid movement image displayed on the display unit at a specific timing after the timing shown in FIG. 16A;

FIG. 17A is a diagram showing an example of the cerebrospinal fluid movement image in a form of expression different from the form of expression shown in FIGS. 15A to 15C; FIG. 17B is a diagram showing an example of the cerebrospinal fluid movement image in a form of expression different from the forms of expression shown in FIGS. 15A to 15C and FIG. 17A;

FIG. 17C is a diagram showing an example of the cerebrospinal fluid movement image in a form of expression different from the forms of expression shown in FIGS. 15A to 15C and FIGS. 17A and 17B;

FIG. 18 is a block diagram of a head tilt motion assistance system according to a second embodiment of the present invention;

FIG. 19 is a diagram showing an example of estimated success degree determination criteria shown in FIG. 18;

FIG. 20 is a diagram showing an example of history information shown in FIG. 18;

FIG. 21 is a flowchart of operation of the head tilt motion assistance system shown in FIG. 18 in a case where the start of the head tilt motion is instructed; and

FIG. 22 is a flowchart of operation of the head tilt motion assistance system shown in FIG. 18 for updating the estimated success degree determination criteria.

### DETAILED DESCRIPTION

Hereinafter, embodiments of the present invention will be described with reference to the drawings.

### First Embodiment

First, a description will be given of a configuration of a head tilt motion assistance system according to a first embodiment of the present invention.

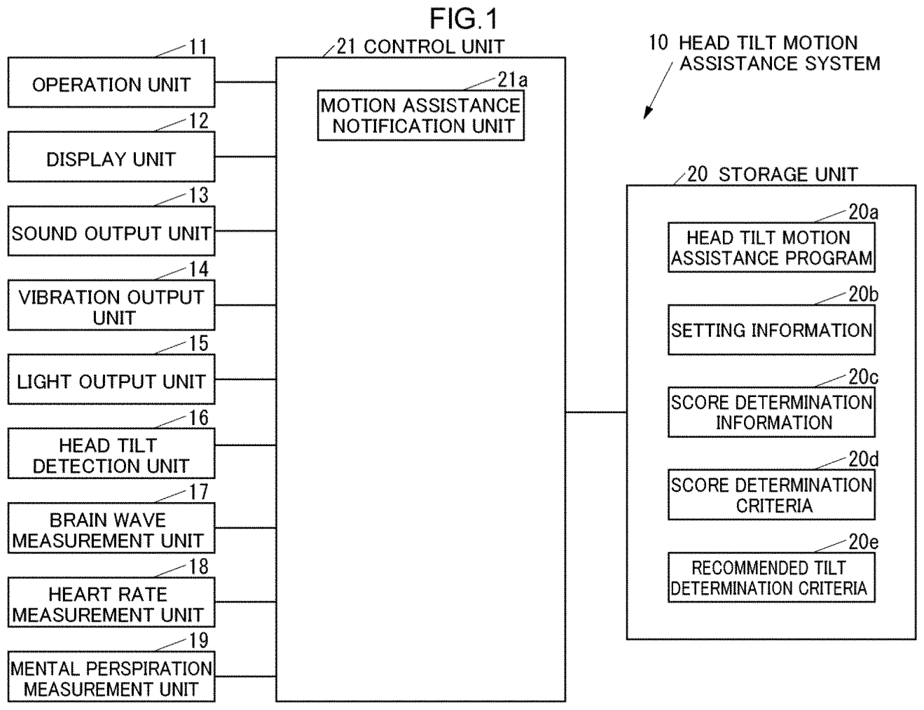

FIG. 1 is a block diagram of a head tilt motion assistance system 10 according to the present embodiment.

As shown in FIG. 1, the head tilt motion assistance system 10 includes an operation unit 11 that is an operation device such as a button to which various operations are input, a display unit 12 that is a display device such as a liquid crystal display (LCD) to display various types of information, a sound output unit 13 that is a sound output device to output various types of information by sound, a vibration output unit 14 that is a vibration output device to output various types of information by vibration, a light output unit 15 such as a glasses-type display device, a head tilt detection unit 16 that detects a direction and an angle of a tilt of a head of a user, a brain wave measurement unit 17 that measures brain waves of the user, a heart rate measurement unit 18 that measures a heart rate of the user, a mental perspiration measurement unit 19 that measures an amount of mental perspiration of the user, a storage unit 20 that is a nonvolatile storage device such as a semiconductor memory to store various types of information, and a control unit 21 that controls the entire head tilt motion assistance system 10.

The sound output unit 13 may include, for example, earphones or headphones. Furthermore, the sound output unit 13 may output sound by bone conduction. The sound output unit 13 is capable of outputting sound (hereinafter, referred to as “γ wave sound”) identical in frequency to a γ wave of the brain waves, so that the sound output unit 13 serves as a γ wave sound output unit according to the present invention.

The light output unit 15 is capable of outputting light (hereinafter, referred to as “γ wave light”) blinking at the same frequency as the frequency of the γ wave of the brain waves, so that the light output unit 15 serves as a γ wave light output unit according to the present invention.

The head tilt detection unit 16 may include, for example, a gyroscope attached to the head of the user.

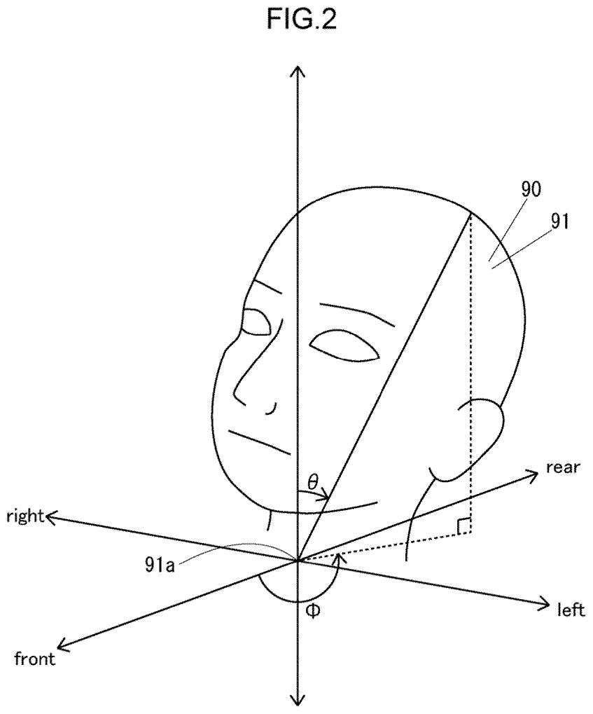

FIG. 2 is a diagram showing a tilt detected by the head tilt detection unit 16.

As shown in FIG. 2, a direction φ of a tilt of a head 91 of a user 90 detected by head tilt detection unit 16 is 0° in a front direction, 90° in a right direction, 180° in a rear direction, and 270° in a left direction.

An angle θ of the tilt of the head 91 of the user 90 detected by the head tilt detection unit 16 is 0° in a case where the head 91 stands straight, 90° in a case where the head 91 lies straight, and 180° in a case where the head 91 stands upside down.

Note that the direction φ is assumed to be 0° when the angle θ is either 0° or 180°.

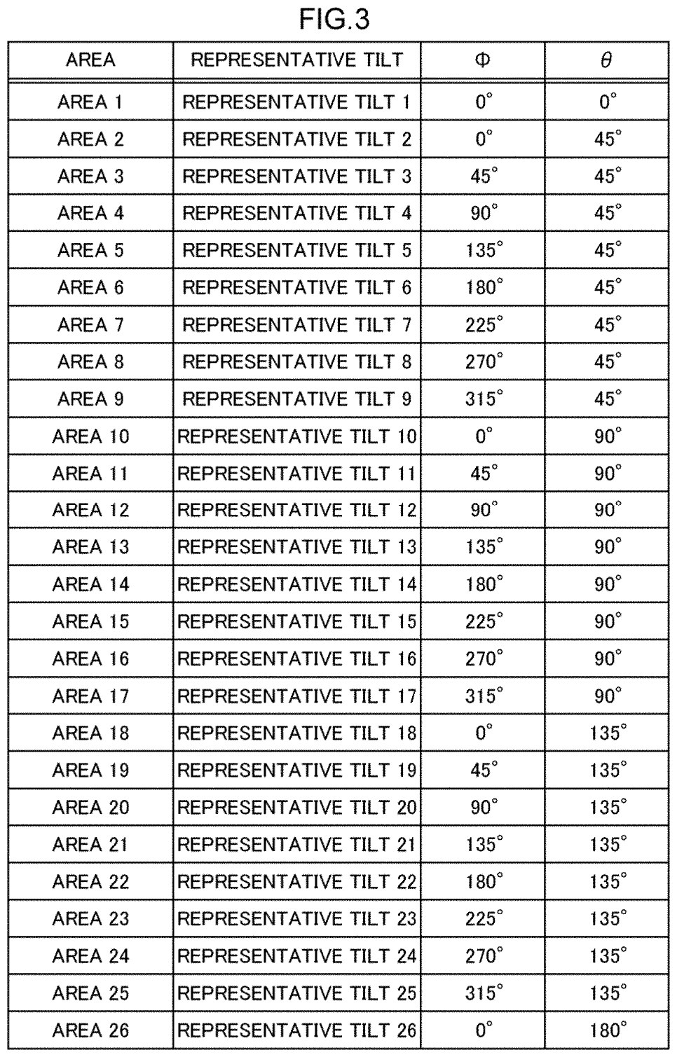

FIG. 3 is a diagram showing a list of areas that may include a tilt detected by the head tilt detection unit 16.

Each area shown in FIG. 3 is obtained by dividing a space into a plurality of areas with a center point 91a (see FIG. 2) of the tilt of the head 91 of the user 90 as a center. FIG. 3 shows a typical tilt (hereinafter, referred to as a “representative tilt”) included in each area. Each area is a set of tilts closest to a corresponding representative tilt in the space. Which of the plurality of areas a tilt on a boundary between the areas belongs is predetermined. The representative tilt is a tilt determined on the basis of a combination of the direction φ in increments of 45° and the angle θ in increments of 45°. For example, a representative tilt of an area 1 is a representative tilt 1 having both the direction φ and the angle θ equal to 0°.

As shown in FIG. 1, the brain wave measurement unit 17 may include, for example, a brain wave measurement device attached to the head of the user.

The heart rate measurement unit 18 may include, for example, a heart rate meter attached to a finger or a wrist of the user.

The mental perspiration measurement unit 19 may include, for example, a perspiration meter attached to a finger, a palm, or a sole of a foot of the user.

The storage unit 20 stores a head tilt motion assistance program 20a for assisting in head tilt motion that is a motion in which the user tilts the head. For example, the head tilt motion assistance program 20a may be installed on the head tilt motion assistance system 10 at a manufacturing stage of the head tilt motion assistance system 10, may be additionally installed on the head tilt motion assistance system 10 from an external storage medium such as a universal serial bus (USB) memory, or may be additionally installed on the head tilt motion assistance system 10 over a network.

The storage unit 20 stores setting information 20b indicating settings of assistance given to the head tilt motion by the head tilt motion assistance system 10.

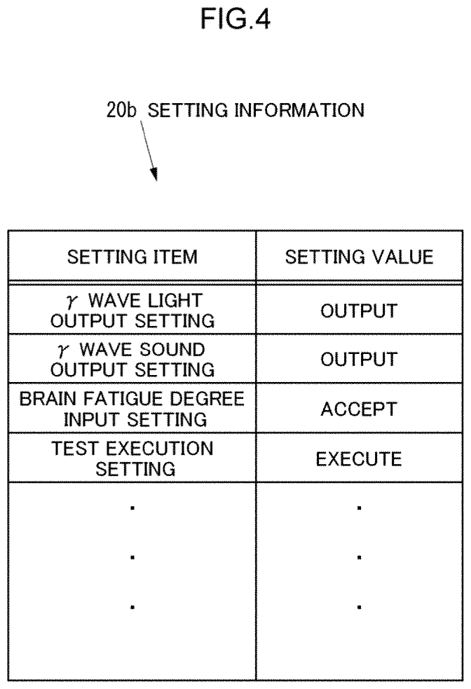

FIG. 4 is a diagram illustrating an example of the setting information 20b.

As shown in FIG. 4, the setting information 20b includes as setting items, for example, a γ wave light output setting indicating a setting as to whether or not to output γ wave light, a γ wave sound output setting indicating a setting as to whether or not to output γ wave sound, a brain fatigue degree input setting indicating a setting as to whether or not to receive input of a fatigue degree of the brain of the user (hereinafter, referred to as a “brain fatigue degree”), and a test execution setting indicating a setting as to whether or not to execute a test for measuring the brain fatigue degree (hereinafter, referred to as a “brain fatigue degree measurement test”).

The brain fatigue degree input by the user (hereinafter, referred to as an “input brain fatigue degree”) may be indicated on ten levels of 1 to 10, for example. The larger a numerical value of the input brain fatigue degree, the more the brain is fatigued.

The brain fatigue degree measured by the brain fatigue degree measurement test (hereinafter, referred to as a “measured brain fatigue degree”) may be indicated on ten levels of 1 to 10, for example. The larger a numerical value of the measured brain fatigue degree, the more the brain is fatigued.

Note that an experiment result showing that the brain fatigue stuff is removed from a brain of a mouse in a case where the γ wave light comes within sight of the mouse, an experiment result showing that the brain fatigue stuff is removed from the brain of the mouse in a case where the γ wave sound comes within hearing of the mouse, and an experiment result showing that the brain fatigue stuff is particularly effectively removed from the brain of the mouse in a case where the γ wave light comes within sight of the mouse and the γ wave sound comes within hearing of the mouse are known. Therefore, even for a human, it is expected that the brain fatigue stuff will be removed from his/her brain in a case where the γ wave light comes within sight of the human or the γ wave sound comes within hearing of the human.

The brain fatigue degree measurement test may be, for example, a test based on the calculation power of the user. For example, the test based on the calculation power of the user may be displaying an addition expression, a subtraction expression, a multiplication expression, or a division expression using numbers having a specific number of digits on the display unit 12 and then determining the brain fatigue degree on the basis of a time taken until a solution to such an expression is input from the operation unit 11 by the user. The brain fatigue degree measurement test may be, for example, a test based on the memory power of the user. For example, the test based on the memory power of the user may be displaying numbers having a specific number of digits only for specific seconds on the display unit 12 and then determining the brain fatigue degree on the basis of the number of correct answers in a case where the numbers are input from the operation unit 11 by the user after a lapse of a specific time. In the brain fatigue degree measurement test, for example, a mean value of user's own past brain fatigue measurement tests may be used as a criterion for comparison of values such as the time for determining the brain fatigue degree and the number of correct answers for determining the brain fatigue degree.

As shown in FIG. 1, the storage unit 20 may store score determination information 20c that is information for determining the score of the head tilt motion.

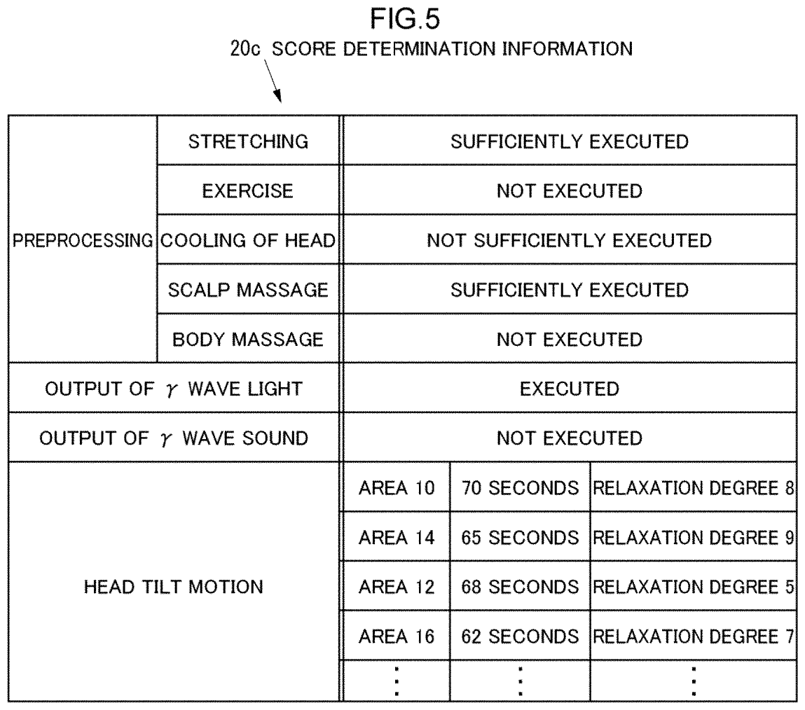

FIG. 5 is a diagram showing an example of the score determination information 20c.

As shown in FIG. 5, the score determination information 20c may include an execution result of preprocessing of the head tilt motion, an execution result of output of the γ wave light, an execution result of output of the γ wave sound, and an execution result of the head tilt motion.

Examples of the preprocessing of the head tilt motion include stretching. When the user makes the stretching, a blood flow rate in the body of the user increases, and the flow rate of the cerebrospinal fluid in the head of the user increases accordingly. Therefore, in a case where the user executes the head tilt motion after the stretching, the brain fatigue stuff such as amyloid β is easily removed from the brain by the cerebrospinal fluid. For example, the score determination information 20c may include an execution result of the stretching in three levels of “not executed”, “not sufficiently executed”, and “sufficiently executed”.

The examples of the preprocessing of the head tilt motion include exercise other than the stretching (hereinafter, simply referred to as “exercise”). When the user makes the exercise, the blood flow rate in the body of the user increases, and the flow rate of the cerebrospinal fluid in the head of the user increases accordingly. Therefore, when the user executes the head tilt motion after the exercise, the brain fatigue stuff is easily removed from the brain by the cerebrospinal fluid. For example, the score determination information 20c may include an execution result of the exercise in three levels of “not executed”, “not sufficiently executed”, and “sufficiently executed”.

The examples of the preprocessing of the head tilt motion include cooling of the head of the user. When the head of the user is cooled, the blood flow rate in the head of the user increases, and the flow rate of cerebrospinal fluid in the head of the user increases accordingly. Therefore, when the user executes the head tilt motion after the cooling of the head of the user, the brain fatigue stuff is easily removed from the brain by the cerebrospinal fluid. For example, the score determination information 20c may include an execution result of the cooling of the head of the user in three levels of “not executed”, “not sufficiently executed”, and “sufficiently executed”.

The examples of the preprocessing of the head tilt motion include massage on a scalp of the user. The scalp massage may be executed, for example, by hand or using an instrument such as a comb or a brush. The scalp massage may include massage on a portion of the head where the scalp exists and acupressure on the portion of the head where the scalp exists. When the scalp of the user is massaged, the blood flow rate in the head of the user increases, and the flow rate of cerebrospinal fluid in the head of the user increases accordingly. Therefore, when the user executes the head tilt motion after the massage on the scalp of the user, the brain fatigue stuff is easily removed from the brain by the cerebrospinal fluid. For example, the score determination information 20c may include an execution result of the massage on the scalp of the user in three levels of “not executed”, “not sufficiently executed”, and “sufficiently executed”.

The examples of the preprocessing of the head tilt motion include massage on the body other than the scalp of the user (hereinafter, simply referred to as a “body”). The body massage may be executed by hand or using an instrument, for example. The body massage may include massage on the body and acupressure on the body. When the body of the user is massaged, the blood flow rate in the head of the user increases, and the flow rate of cerebrospinal fluid in the head of the user increases accordingly. Therefore, when the user executes the head tilt motion after the massage on the body of the user, the brain fatigue stuff is easily removed from the brain by the cerebrospinal fluid. For example, the score determination information 20c may include an execution result of the massage on the body of the user in three levels of “not executed”, “not sufficiently executed”, and “sufficiently executed”.

The score determination information 20c may include either “not executed” or “executed” as the execution result of the output of the γ wave light.

The score determination information 20c may include either “not executed” or “executed” as the execution result of the output of the γ wave sound.

The score determination information 20c may include, as the execution result of the head tilt motion, an execution result of a motion of tilting the head and bringing the head to rest (hereinafter, referred to as a “tilt and stop motion”) in order of execution time of the tilt and stop motion. The execution result of the tilt and stop motion includes an area where the tilt of the head of the user exists at the time of stopping a change in the tilt of the head of the user (hereinafter, referred to as a “stop of tilt change”) in the tilt and stop motion, duration of the stop of tilt change in the tilt and stop motion, and a degree of relaxation (hereinafter, referred to as a “relaxation degree”) of the user in the tilt and stop motion.

The relaxation degree may be indicated on ten levels of 1 to 10, for example. The larger a numerical value of the relaxation degree, the more the brain is relaxed. It is known that the more the brain is relaxed, the more smoothly the cerebrospinal fluid flows in the brain.

As shown in FIG. 1, the storage unit 20 may store score determination criteria 20d that are criteria for determining the score of the head tilt motion.

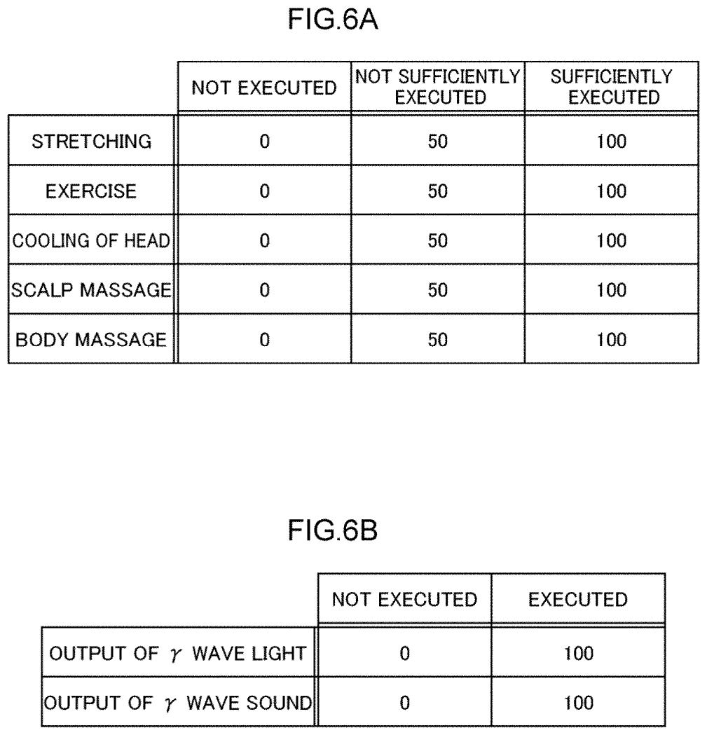

FIG. 6A is a diagram showing an example of a criterion for the score of the preprocessing of the head tilt motion among the criteria included in the score determination criteria 20d. FIG. 6B is a diagram showing an example of a criterion for the score of the output of the γ wave light and the γ wave sound among the criteria included in the score determination criteria 20d.

In the criterion shown in FIG. 6A, for each of “stretching”, “exercise”, “cooling of head”, “scalp massage”, and “body massage”, a score in a case where no exercise is executed is set at 0 points, a score in a case where the exercise is executed but not sufficiently is set at 50 points, and a score in a case where the exercise is sufficiently executed is set at 100 points. The criterion shown in FIG. 6A is a criterion according to an execution detail of the preprocessing of the head tilt motion. In the criterion shown in FIG. 6A, a range consisting of all of degree that a degree of execution of the preprocessing of the head tilt motion can be is divided into three levels of “not executed”, “not sufficiently executed”, and “sufficiently executed”. The criterion shown in FIG. 6A is a criterion according to the degree of the execution of the preprocessing of the head tilt motion. The criterion shown in FIG. 6A is a criterion in which higher score corresponds to a level associated with a higher degree of the execution of the preprocessing of the head tilt motion in a case where a type of the preprocessing of the head tilt motion is constant. For example, in the criterion shown in FIG. 6A, in a case where the type of the preprocessing of the head tilt motion is “stretching”, a score to which a level at which the degree of the execution of the preprocessing of the head tilt motion is “not executed” is corresponded is set at 0 points, a score to which a level at which the degree of the execution of the preprocessing of the head tilt motion is “not sufficiently executed” is corresponded is set at 50 points, and a score to which a level at which the degree of the execution of the preprocessing of the head tilt motion is “sufficiently executed” is corresponded is set at 100 points. In the criterion shown in FIG. 6A, a range consisting of all of quantity that a quantity of a type of an executed preprocessing can be is divided into six levels of 0, 1, 2, 3, 4 and 5. The criterion shown in FIG. 6A is a criterion according to the quantity of the type of the executed preprocessing. The criterion shown in FIG. 6A is a criterion in which higher score corresponds to a level associated with a larger quantity of the type of the executed preprocessing in a case where the degree of the execution of the executed preprocessing is constant. For example, in the criterion shown in FIG. 6A, in a case where the degree of the execution of the executed preprocessing is “sufficiently executed”, a score to which a level at which the quantity of the type of the executed preprocessing is 0 is corresponded is set at 0 points, a score to which a level at which the quantity of the type of the executed preprocessing is 1 is corresponded is set at 100 points, a score to which a level at which the quantity of the type of the executed preprocessing is 2 is corresponded is set at 200 points, a score to which a level at which the quantity of the type of the executed preprocessing is 3 is corresponded is set at 300 points, a score to which a level at which the quantity of the type of the executed preprocessing is 4 is corresponded is set at 400 points, a score to which a level at which the quantity of the type of the executed preprocessing is 5 is corresponded is set at 500 points.

In the criterion shown in FIG. 6B, for each of “output of the γ wave light” and “output of the γ wave sound”, a score in a case where no output is executed is set as 0 points, and a score in a case where the output is executed is set at 100 points.

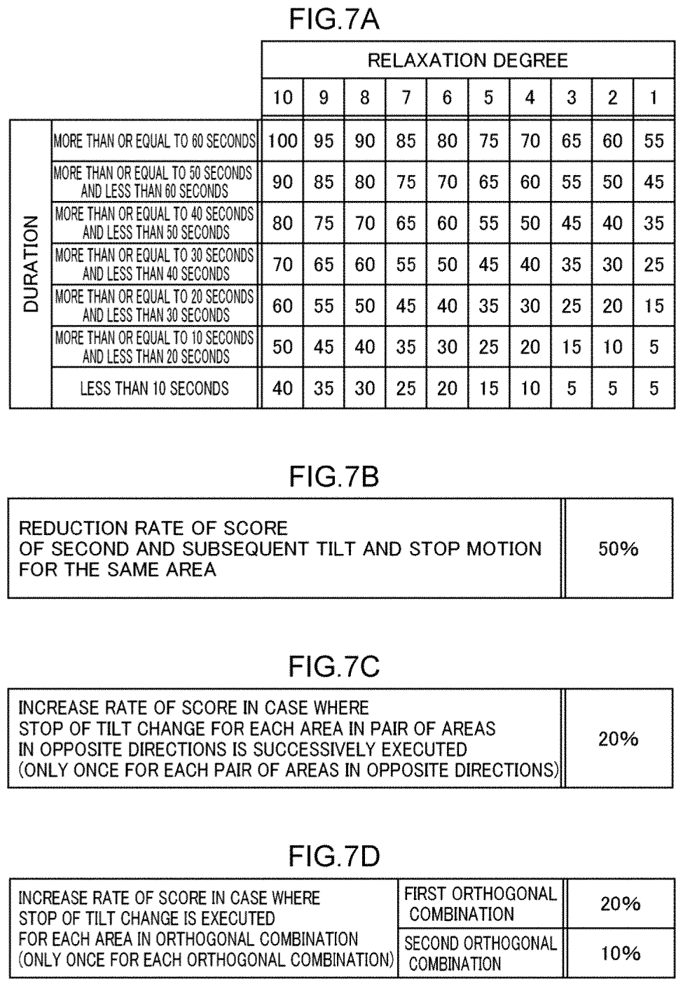

FIG. 7A is a diagram showing an example of a criterion for the score of the tilt and stop motion among the criteria included in the score determination criteria 20d. FIG. 7B is a diagram showing an example of a criterion for a reduction rate of a score of the second and subsequent tilt and stop motions for the same area among the criteria included in the score determination criteria 20d. FIG. 7C is a diagram showing an example of a criterion for an increase rate of a score in a case where the stop of tilt change is successively executed on each area in a pair of areas present in directions opposite to each other with respect to the center point 91a (hereinafter, referred to as a “a pair of areas in opposite directions”) among the criteria included in the score determination criteria 20d. FIG. 7D is a diagram showing an example of a criterion for an increase rate of a score in a case where the stop of tilt change is executed for each area in a combination of a pair of areas in opposite directions orthogonal to each other (hereinafter, referred to as an “orthogonal combination”) among the criteria included in the score determination criteria 20d.

In the criterion shown in FIG. 7A, a score is set for a combination of the duration of the stop of tilt change and the relaxation degree. The criterion shown in FIG. 7A is a criterion based on the duration of the stop of tilt change of the head of the user in the head tilt motion. In the criterion shown in FIG. 7A, a range consisting of all of time that the duration of the stop of tilt change of the head of the user in the head tilt motion can be is divided into seven levels of “less than 10 seconds”, “more than or equal to 10 seconds and less than 20 seconds”, “more than or equal to 20 seconds and less than 30 seconds”, “more than or equal to 30 seconds and less than 40 seconds”, “more than or equal to 40 seconds and less than 50 seconds”, “more than or equal to 50 seconds and less than 60 seconds”, and “more than or equal to 60 seconds”. The criterion shown in FIG. 7A is a criterion in which higher score corresponds to a level associated with a longer duration of the stop of tilt change of the head of the user in the head tilt motion in a case where the relaxation degree is constant. For example, in the criterion shown in FIG. 7A, in a case where the relaxation degree is 10, a score to which a level at which the duration of the stop of tilt change of the head of the user in the head tilt motion is “less than 10 seconds” is corresponded is set at 40 points, a score to which a level at which the duration of the stop of tilt change of the head of the user in the head tilt motion is “more than or equal to 10 seconds and less than 20 seconds” is corresponded is set at 50 points, a score to which a level at which the duration of the stop of tilt change of the head of the user in the head tilt motion is “more than or equal to 20 seconds and less than 30 seconds” is corresponded is set at 60 points, a score to which a level at which the duration of the stop of tilt change of the head of the user in the head tilt motion is “more than or equal to 30 seconds and less than 40 seconds” is corresponded is set at 70 points, a score to which a level at which the duration of the stop of tilt change of the head of the user in the head tilt motion is “more than or equal to 40 seconds and less than 50 seconds” is corresponded is set at 80 points, a score to which a level at which the duration of the stop of tilt change of the head of the user in the head tilt motion is “more than or equal to 50 seconds and less than 60 seconds” is corresponded is set at 90 points, and a score to which a level at which the duration of the stop of tilt change of the head of the user in the head tilt motion is “more than or equal to 60 seconds” is corresponded is set at 100 points. Further, the criterion shown in FIG. 7A is a criterion based on the relaxation degree of the user in the head tilt motion. In the criterion shown in FIG. 7A, a range consisting of all of degree that the relaxation degree of the user in the head tilt motion can be is divided into ten levels of 1 to 10. The criterion shown in FIG. 7A is a criterion in which higher score corresponds to a level associated with a higher relaxation degree of the user in the head tilt motion in a case where the duration of the stop of tilt change of the head of the user in the head tilt motion is constant. For example, in the criterion shown in FIG. 7A, in a case where the duration of the stop of tilt change of the head of the user in the head tilt motion is “more than or equal to 60 seconds”, a score to which a level at which the relaxation degree of the user in the head tilt motion is 1 is corresponded is set at 55 points, a score to which a level at which the relaxation degree of the user in the head tilt motion is 2 is corresponded is set at 60 points, a score to which a level at which the relaxation degree of the user in the head tilt motion is 3 is corresponded is set at 65 points, a score to which a level at which the relaxation degree of the user in the head tilt motion is 4 is corresponded is set at 70 points, a score to which a level at which the relaxation degree of the user in the head tilt motion is 5 is corresponded is set at 75 points, a score to which a level at which the relaxation degree of the user in the head tilt motion is 6 is corresponded is set at 80 points, a score to which a level at which the relaxation degree of the user in the head tilt motion is 7 is corresponded is set at 85 points, a score to which a level at which the relaxation degree of the user in the head tilt motion is 8 is corresponded is set at 90 points, a score to which a level at which the relaxation degree of the user in the head tilt motion is 9 is corresponded is set at 95 points, and a score to which a level at which the relaxation degree of the user in the head tilt motion is 10 is corresponded is set at 100 points.

In the criterion shown in FIG. 7B, the reduction rate of the score of the second and subsequent stop of tilt change for the same area is set at 50%.

In the criterion shown in FIG. 7C, the increase rate of the score in a case where the stop of tilt change is successively executed on each area in the pair of areas in opposite directions is set at 20%. This increase is executed only once on each of the pair of areas in opposite directions. The criterion shown in FIG. 7C is a criterion for increasing the score of the head tilt motion when, in a case where the stop of tilt change is executed in a state where the tilt of the head exists in a first area, the stop of tilt change executed immediately after the stop of tilt change executed in a state where the tilt of the head exists in the first area is the stop of tilt change executed in a state where the tilt of the head of the user exists in a second area existing in the direction opposite to the first area. The second area exists on a side opposite to the first area with respect to the center point of the tilt of the head of the user. Note that the pair of areas in opposite directions includes a pair of an area 1 and an area 26, a pair of an area 2 and an area 22, a pair of an area 3 and an area 23, a pair of an area 4 and an area 24, a pair of an area 5 and an area 25, a pair of an area 6 and an area 18, a pair of an area 7 and an area 19, a pair of an area 8 and an area 20, a pair of an area 9 and an area 21, a pair of an area 10 and an area 14, a pair of an area 11 and an area 15, a pair of an area 12 and an area 16, and a pair of an area 13 and an area 17.

In the criterion shown in FIG. 7D, the increase rate of the score in a case where the stop of tilt change is executed for each area in the orthogonal combination is set at 20% for the first orthogonal combination, and is set at 10% for the second and subsequent orthogonal combinations. This increase is executed only once on each orthogonal combination. The criterion shown in FIG. 7D is a criterion for increasing the score of the head tilt motion in a case where the stop of tilt change of the head of the user is executed in the head tilt motion in all a state where the tilt of the head of the user exists in the first area, a state where the tilt of the head of the user exists in the second area existing in the direction opposite to the first area, a state where the tilt of the head of the user exists in a third area existing in the direction orthogonal to the second area, and a state where the tilt of the head of the user exists in a fourth area existing in the direction opposite to the third area. The second area exists on a side opposite to the first area with respect to the center point of the tilt of the head of the user. The third area exists with 90° shift from the second area around the center point of the tilt of the head of the user. The fourth area exists on a side opposite to the third area with respect to the center point of the tilt of the head of the user. Note that the orthogonal combinations to be subjected to an increase in score include a combination of a pair of the area 10 and the area 14 and a pair of the area 12 and the area 16 orthogonal to the pair of the area 10 and the area 14. The orthogonal combinations to be subjected to an increase in score include a combination of a pair of the area 11 and the area 15 and a pair of the area 13 and the area 17 orthogonal to the pair of the area 11 and the area 15.

The criteria shown in FIGS. 7A to 7D are criteria according to the execution detail of the head tilt motion.

As shown in FIG. 1, the storage unit 20 may store criteria 20e for determining a recommended tilt of the stop of tilt change (hereinafter, referred to as “recommended tilt determination criteria”). The recommended tilt determination criteria 20e may include, for example, a criterion that the recommended tilt of the first stop of tilt change in the head tilt motion is a representative tilt 10. Further, for the recommended tilt of the second and subsequent stop of tilt change in the head tilt motion, the recommended tilt determination criteria 20e may include, for example, a criterion for determining a representative tilt of an area (hereinafter, referred to as a “second area” in this paragraph) existing in a direction opposite to any area (hereinafter, referred to as a “first area” in this paragraph) as the recommended tilt of the stop of tilt change when, in case where the stop of tilt change is executed in a state where the tilt of the head of the user exists in the first area, the stop of tilt change executed immediately before the stop of tilt change executed in a state where the tilt of the head of the user exists in the first area is not the stop of tilt change executed in a state where the tilt of the head of the user exists in the second area. Further, for the recommended tilt of the second and subsequent stop of tilt change in the head tilt motion, the recommended tilt determination criteria 20e may include, for example, a criterion for determining a representative tilt of an area existing in a direction orthogonal to the second area as the recommended tilt of the stop of tilt change, when, in a case where the stop of tilt change is executed in a state where the tilt of the head of the user exists in the first area, the stop of tilt change executed immediately before the stop of tilt change executed in a state where the tilt of the head of the user exists in the first area is the stop of tilt change executed in a state where the tilt of the head of the user exists in the second area.

The control unit 21 shown in FIG. 1 includes, for example, a central processing unit (CPU), a read only memory (ROM) that stores a program and various data, and a random access memory (RAM) as a memory used as a work area of the CPU of the control unit 21. The CPU of the control unit 21 executes a program stored in the storage unit 20 or the ROM of the control unit 21.

The control unit 21 may include, for example, a computer such as a smartphone, a tablet, a wearable device, a personal computer (PC), or a cloud server. In a case where the control unit 21 includes a computer such as a smartphone, a tablet, a wearable device, or a PC, the computer included in the control unit 21 may also serve as at least a part of the operation unit 11, the display unit 12, the sound output unit 13, the vibration output unit 14, and the storage unit 20.

The control unit 21 executes the head tilt motion assistance program 20a to implement a motion assistance notification unit 21a that makes a motion assistance notification for assisting in the head tilt motion.

Next, the operation of the head tilt motion assistance system 10 will be described.

First, the operation of the head tilt motion assistance system 10 for updating the setting information 20b will be described.

The user can instruct, via the operation unit 11, the head tilt motion assistance system 10 to update the setting information 20b. In order to make the instruction to update the setting information 20b, the user designates a setting value for a setting item to be updated in the setting information 20b via the operation unit 11. Upon receipt of the instruction to update the setting information 20b, the motion assistance notification unit 21a updates the setting information 20b with the setting value designated by the user.

Next, the operation of the head tilt motion assistance system 10 upon receipt of an instruction to start the head tilt motion will be described.

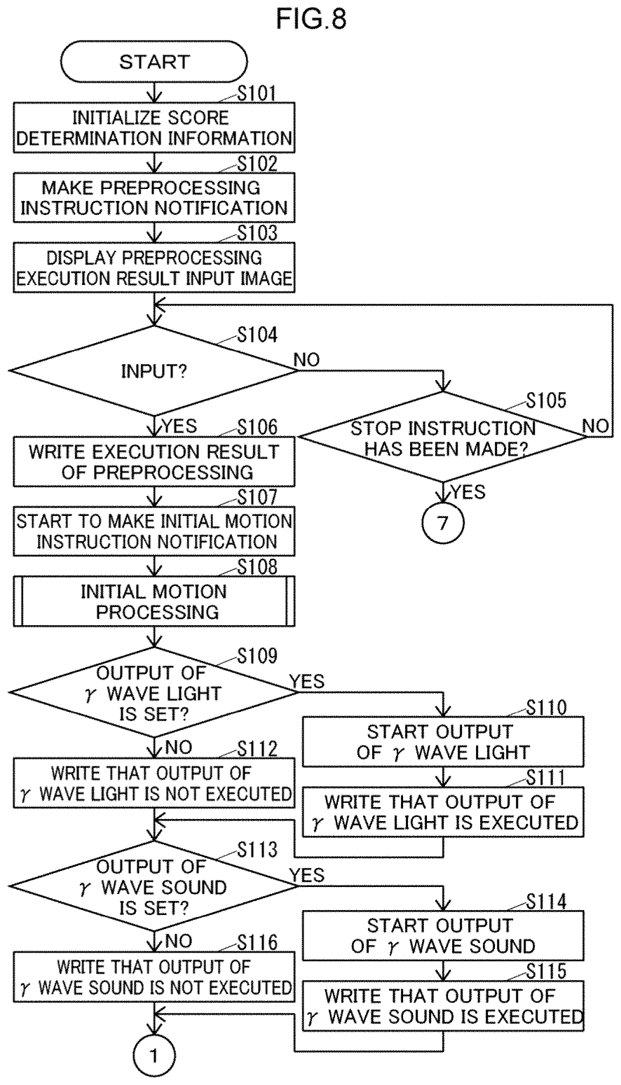

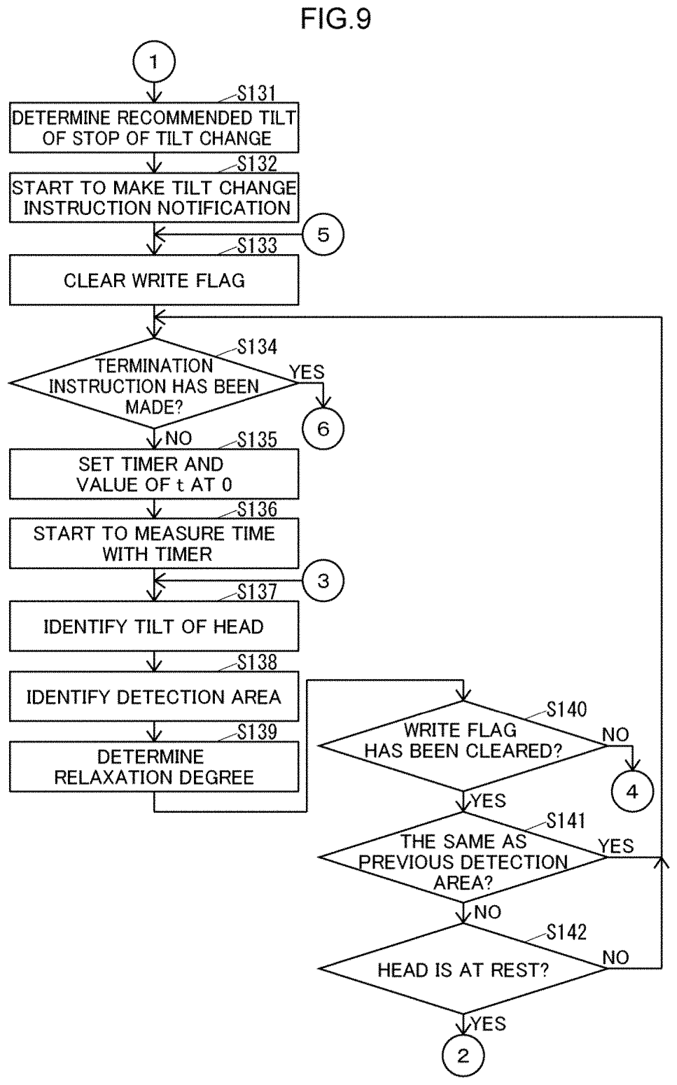

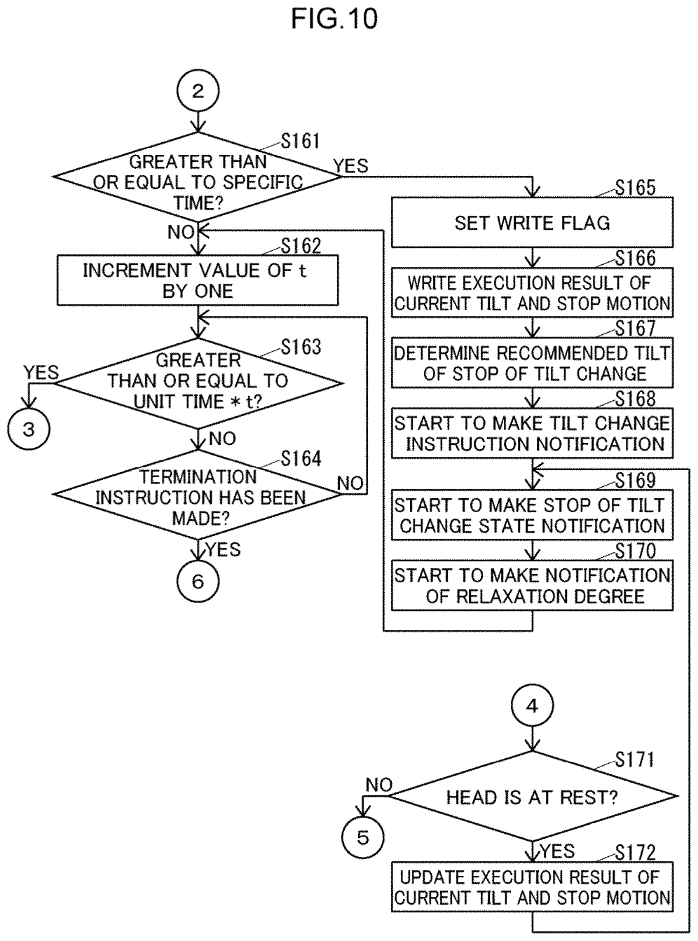

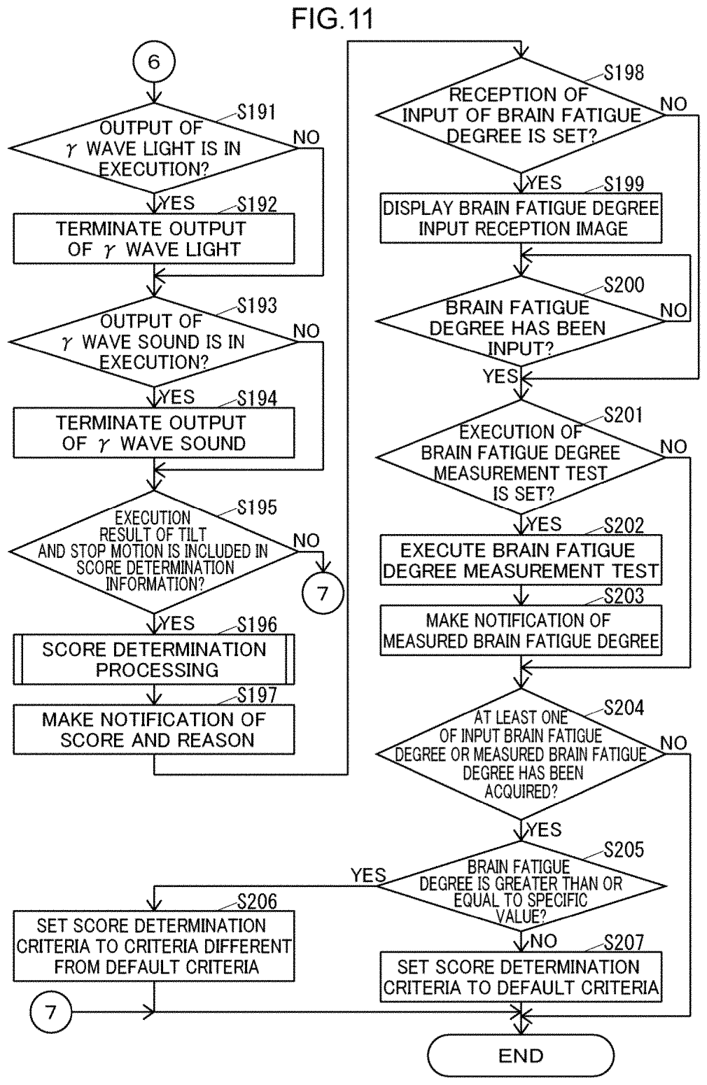

FIG. 8 is a flowchart of a part of the operation of the head tilt motion assistance system 10 upon receipt of the instruction to start the head tilt motion. FIG. 9 is a flowchart of operation subsequent to the operation illustrated in FIG. 8. FIG. 10 is a flowchart of operation subsequent to the operation illustrated in FIG. 9. FIG. 11 is a flowchart of operation subsequent to the operation illustrated in FIG. 10.

In order to start the head tilt motion, the user can instruct, via the operation unit 11, the head tilt motion assistance system 10 to start the head tilt motion. Upon receipt of the instruction to start the head tilt motion, the motion assistance notification unit 21a executes the operation shown in FIGS. 8 to 11.

As shown in FIGS. 8 to 11, the motion assistance notification unit 21a initializes the score determination information 20c (S101). Therefore, the score determination information 20c is void information including no value.

Upon completion of the processing of S101, the motion assistance notification unit 21a makes a notification for instructing the user to make stretching, exercise, cooling of the head of the user, scalp massage, and body massage (hereinafter, referred to as a “preprocessing instruction notification”), for example, by at least one of display by the display unit 12, sound output by the sound output unit 13, or vibration output by the vibration output unit 14 (S102). This allows the user who has received the preprocessing instruction notification to make the stretching, the exercise, the cooling of the head of the user, scalp massage, and the body massage.

Upon completion of the processing of S102, the motion assistance notification unit 21a displays, on the display unit 12, an image for inputting execution results of the stretching, the exercise, the cooling of the head of the user, the scalp massage, and the body massage (hereinafter, referred to as a “preprocessing execution result input image”) (S103). The user can input, via, for example, the operation unit 11, the execution results of the stretching, the exercise, the cooling of the head of the user, the scalp massage, and the body massage to the preprocessing execution result input image. For example, in the preprocessing execution result input image, each execution result is input in three levels of “not executed”, “not sufficiently executed”, and “sufficiently executed” for the stretching, the exercise, the cooling of the head of the user, the scalp massage, and the body massage.

Upon completion of the processing of S103, the motion assistance notification unit 21a determines whether or not all the execution results of the stretching, the exercise, the cooling of the head of the user, the scalp massage, and the body massage have been input to the preprocessing execution result input image (S104).

Upon determination in S104 that at least one of the stretching, the exercise, the cooling of the head of the user, the scalp massage, or the body massage has not been input to the preprocessing execution result input image, the motion assistance notification unit 21a determines whether or not the instruction to stop the head tilt motion has been made (S105). The user can instruct, via the operation unit 11, the head tilt motion assistance system 10 to stop the head tilt motion.

Upon determination in S105 that the instruction to stop the head tilt motion has been made, the motion assistance notification unit 21a terminates the operation shown in FIGS. 8 to 11.

Upon determination in S105 that the instruction to stop the head tilt motion has not been made, the motion assistance notification unit 21a executes the processing of S104.

Upon determination in S104 that all the execution results of the stretching, the exercise, the cooling of the head of the user, the scalp massage, and the body massage have been input to the preprocessing execution result input image, the motion assistance notification unit 21a writes the execution results input to the preprocessing execution result input image to the score determination information 20c (S106).

Upon completion of the processing of S106, the motion assistance notification unit 21a starts to make a notification (hereinafter, referred to as “initial motion instruction notification”) for instructing the user to execute a motion (hereinafter referred to as an “initial motion”) of setting the head in an initial state, that is, a state where the head stands straight and comes to rest, for example, by at least one of display by the display unit 12, sound output by the sound output unit 13, or vibration output by the vibration output unit 14 (S107). This allows the user who has received the initial motion instruction notification in S107 to stand the head straight and bring the head to rest.

Upon completion of the processing of S107, the motion assistance notification unit 21a executes processing of causing the user to execute the initial motion (hereinafter, referred to as “initial motion processing”) (S108).

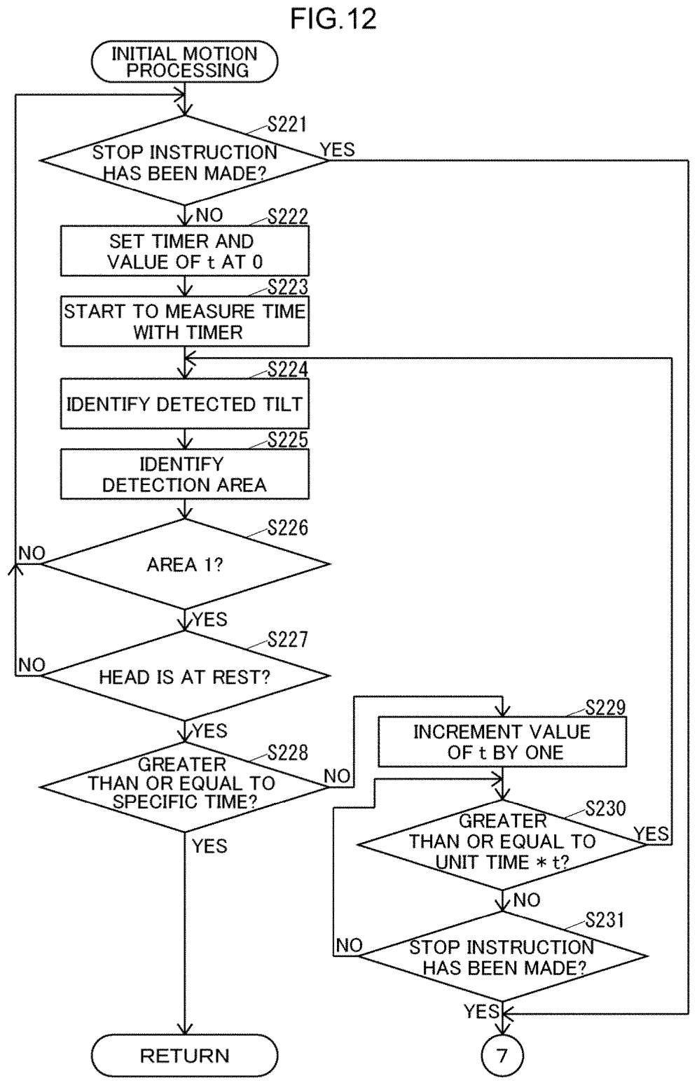

FIG. 12 is a flowchart of the initial motion processing shown in FIGS. 8 to 11.

As shown in FIG. 12, the motion assistance notification unit 21a determines whether or not the instruction to stop the head tilt motion has been made (S221). The user can instruct, via the operation unit 11, the head tilt motion assistance system 10 to stop the head tilt motion.

Upon determination in S221 that the instruction to stop the head tilt motion has been made, the motion assistance notification unit 21a terminates the operation shown in FIGS. 8 to 11.

Upon determination in S221 that the instruction to stop the head tilt motion has not been made, the motion assistance notification unit 21a sets both a value of a timer and a value of a variable t at 0 (S222).

Upon completion of the processing of S222, the motion assistance notification unit 21a starts to measure time with the timer (S223).

Upon completion of the processing of S223, the motion assistance notification unit 21a identifies the tilt of the head of the user detected by the head tilt detection unit 16 (S224).

Upon completion of the processing of S224, the motion assistance notification unit 21a identifies an area including the tilt identified in S224 (hereinafter, referred to as a “detection area”) among the areas shown in FIG. 3 (S225).

Upon completion of the processing of S225, the motion assistance notification unit 21a determines whether or not the detection area identified in S225 is an area corresponding to a case where the head stands straight, that is, the area 1 (S226).

Upon determination in S226 that the detection area identified in S225 is not the area 1, the motion assistance notification unit 21a executes the processing of S221.

Upon determination in S226 that the detection area identified in S225 is the area 1, the motion assistance notification unit 21a determines whether or not the head is at rest (S227). As a method for determining whether or not the head is at rest, various methods are applicable. The motion assistance notification unit 21a always determines that the head is at rest in the first execution of S227 after the last execution of the processing of S222. For example, the motion assistance notification unit 21a may determine that the head is at rest when a deviation between all the tilts identified in S224 after the last execution of the processing of S222 falls within a specific range of the space shown in FIG. 2 in the second and subsequent execution of S227 after the last execution of the processing of S222.

Upon determination in S227 that the head is not at rest, the motion assistance notification unit 21a executes the processing of S221.

Upon determination in S227 that the head is at rest, the motion assistance notification unit 21a determines whether or not the time measured by the timer is greater than or equal to a specific time (S228). The specific time in S228 is a preset time, for example, 5 seconds.

Upon determination in S228 that the time measured by the timer is not greater than or equal to the specific time, the motion assistance notification unit 21a increments the value of the variable t by one (S229).

Upon completion of the processing of S229, the motion assistance notification unit 21a determines whether or not the time measured by the timer is greater than or equal to a time that is a product of a specific unit time and the value of the variable t (S230). The specific unit time in S230 is a preset time, for example, 0.1 seconds.

Upon determination in S230 that the time measured by the timer is not greater than or equal to the time that is the product of the specific unit time and the value of the variable t, the motion assistance notification unit 21a determines whether or not the instruction to stop the head tilt motion has been made (S231). The user can instruct, via the operation unit 11, the head tilt motion assistance system 10 to stop the head tilt motion.

Upon determination in S231 that the instruction to stop the head tilt motion has been made, the motion assistance notification unit 21a terminates the operation shown in FIGS. 8 to 11.

Upon determination in S231 that the instruction to stop the head tilt motion has not been made, the motion assistance notification unit 21a executes the processing of S230.

Upon determination in S230 that the time measured by the timer is greater than or equal to the time that is the product of the specific unit time and the value of the variable t, the motion assistance notification unit 21a executes the processing of S224.

Upon determination in S228 that the time measured by the timer is greater than or equal to the specific time, the motion assistance notification unit 21a terminates the initial motion processing shown in FIG. 12.

As shown in FIGS. 8 to 11, upon completion of the initial motion processing of S108, the motion assistance notification unit 21a determines whether or not the output of the γ wave light is set in the γ wave light output setting of the setting information 20b (S109).

Upon determination in S109 that the output of the γ wave light is set in the γ wave light output setting of the setting information 20b, the motion assistance notification unit 21a starts the output of the γ wave light by the light output unit 15 (S110). This brings the γ wave light output by the light output unit 15 within sight of the user.

Upon completion of the processing of S110, the motion assistance notification unit 21a writes that the γ wave light has been output to the score determination information 20c (S111).

Upon determination in S109 that the output of the γ wave light is not set in the γ wave light output setting of the setting information 20b, the motion assistance notification unit 21a writes that the γ wave light has not been output to the score determination information 20c (S112).

Upon completion of the processing of S111 or S112, the motion assistance notification unit 21a determines whether or not the output of the γ wave sound is set in the γ wave sound output setting of the setting information 20b (S113).

Upon determination in S113 that the output of the γ wave sound is set in the γ wave sound output setting of the setting information 20b, the motion assistance notification unit 21a starts the output of the γ wave sound by the sound output unit 13 (S114). This brings the γ wave sound output by the sound output unit 13 within hearing of the user.

Upon completion of the processing of S114, the motion assistance notification unit 21a writes that the γ wave sound has been output to the score determination information 20c (S115).

Upon determination in S113 that the output of the γ wave sound is not set in the γ wave sound output setting of the setting information 20b, the motion assistance notification unit 21a writes that the γ wave sound has not been output to the score determination information 20c (S116).

Upon completion of the processing of S115 or S116, the motion assistance notification unit 21a determines the recommended tilt of the stop of tilt change on the basis of the recommended tilt determination criteria 20e and the execution result of the head tilt motion indicated in the score determination information 20c (S131).

Upon completion of the processing of S131, the motion assistance notification unit 21a starts to make a notification for instructing to change the head of the user to the recommended tilt (hereinafter, referred to as a “tilt change instruction notification”) by, for example, at least one of display by the display unit 12, sound output by the sound output unit 13, or vibration output by the vibration output unit 14 (S132). The motion assistance notification unit 21a generates, as the tilt change instruction notification that starts to be made in S132, a notification for instructing to move the head of the user to the tilt determined in S131 and bring the head to rest. This allows the user who has received the tilt change instruction notification to move the head to the tilt indicated by the tilt change instruction notification and bring the head to rest.

Upon completion of the processing of S132, the motion assistance notification unit 21a clears a write flag that is a flag for determining whether or not to write the execution result of the head tilt motion to the score determination information 20c (S133).

Upon completion of the processing of S133, the motion assistance notification unit 21a determines whether or not the instruction to terminate the head tilt motion has been made (S134). The user can instruct, via the operation unit 11, the head tilt motion assistance system 10 to terminate the head tilt motion.

Upon determination in S134 that the instruction to terminate the head tilt motion has not been made, the motion assistance notification unit 21a sets both the value of the timer and the value of the variable t at 0 (S135).

Upon completion of the processing of S135, the motion assistance notification unit 21a starts to measure the time with the timer (S136).

Upon completion of the processing of S136, the motion assistance notification unit 21a identifies the tilt of the head of the user detected by the head tilt detection unit 16 (S137).

Upon completion of the processing of S137, the motion assistance notification unit 21a identifies a detection area that is an area including the tilt identified in S137, as with the processing of S225 (S138).

Upon completion of the processing of S138, the motion assistance notification unit 21a determines the relaxation degree on the basis of the brain waves measured by the brain wave measurement unit 17, the heart rate measured by the heart rate measurement unit 18, and the amount of mental perspiration measured by the mental perspiration measurement unit 19 (S139).

For example, in a case where the heart rate measured by the heart rate measurement unit 18 and the amount of mental perspiration measured by the mental perspiration measurement unit 19 are constant, the motion assistance notification unit 21a determines that the relaxation degree is higher when the brain waves measured by the brain wave measurement unit 17 are α waves than when the brain waves are β waves. For example, in a case where the heart rate measured by the heart rate measurement unit 18 and the amount of mental perspiration measured by the mental perspiration measurement unit 19 are constant, the motion assistance notification unit 21a determines that the relaxation degree is higher when the brain waves measured by the brain wave measurement unit 17 are θ waves than when the brain waves are α waves. For example, in a case where the heart rate measured by the heart rate measurement unit 18 and the amount of mental perspiration measured by the mental perspiration measurement unit 19 are constant, the motion assistance notification unit 21a determines that the relaxation degree is higher when the brain waves measured by the brain wave measurement unit 17 are δ waves than when the brain waves are θ waves. For example, in a case where the heart rate measured by the heart rate measurement unit 18 and the amount of mental perspiration measured by the mental perspiration measurement unit 19 are constant, the motion assistance notification unit 21a determines that the relaxation degree is higher when the brain waves measured by the brain wave measurement unit 17 are γ waves than when the brain waves are δ waves.

For example, in a case where the brain waves measured by the brain wave measurement unit 17 and the amount of mental perspiration measured by the mental perspiration measurement unit 19 are constant, the motion assistance notification unit 21a determines that the relaxation degree is higher when the heart rate measured by the heart rate measurement unit 18 is low than when the heart rate is high.

For example, in a case where the brain waves measured by the brain wave measurement unit 17 and the heart rate measured by the heart rate measurement unit 18 are constant, the motion assistance notification unit 21a determines that the relaxation degree is higher when the amount of mental perspiration measured by the mental perspiration measurement unit 19 is small than when the amount of mental perspiration is large.

Upon completion of the processing of S139, the motion assistance notification unit 21a determines whether or not the write flag has been cleared (S140).

Upon determination in S140 that the write flag has been cleared, the motion assistance notification unit 21a determines whether or not the detection area identified in S138 is the same as the previous detection area (S141). Here, in a case where no area has been written to the execution result of the head tilt motion indicated in the score determination information 20c, the motion assistance notification unit 21a treats the area 1 as the previous detection area. On the other hand, in a case where an area has been already written to the execution result of the head tilt motion indicated in the score determination information 20c, the motion assistance notification unit 21a treats the latest area written to the execution result of the head tilt motion indicated in the score determination information 20c as the previous detection area.

Upon determination in S141 that the detection area identified in S138 is the same as the previous detection area, the motion assistance notification unit 21a executes the processing of S134.

Upon determination in S141 that the detection area identified in S138 is different from the previous detection area, the motion assistance notification unit 21a determines whether or not the head is at rest (S142). As a method for determining whether or not the head is at rest, various methods are applicable. The motion assistance notification unit 21a always determines that the head is at rest in the first execution of S142 after the last execution of the processing of S135. For example, the motion assistance notification unit 21a may determine that the head is at rest in a case where a deviation between all the tilts identified in S137 after the last execution of the processing of S135 falls within a specific range of the space shown in FIG. 2 in the second and subsequent execution of S142 after the last execution of the processing of S135.

Upon determination in S142 that the head is not at rest, the motion assistance notification unit 21a executes the processing of S134.

Upon determination in S142 that the head is at rest, the motion assistance notification unit 21a determines whether or not the time measured by the timer is greater than or equal to a specific time (S161). The specific time in S161 is a preset time, for example, 5 seconds.

Upon determination in S161 that the time measured by the timer is not greater than or equal to the specific time, the motion assistance notification unit 21a increments the value of the variable t by one (S162).

Upon completion of the processing of S162, the motion assistance notification unit 21a determines whether or not the time measured by the timer is greater than or equal to a time that is a product of a specific unit time and the value of the variable t (S163). The specific unit time in S163 is a preset time, for example, 0.1 seconds.

Upon determination in S163 that the time measured by the timer is not greater than or equal to the time that is the product of the specific unit time and the value of the variable t, the motion assistance notification unit 21a determines whether or not the instruction to terminate the head tilt motion has been made (S164). The user can instruct, via the operation unit 11, the head tilt motion assistance system 10 to terminate the head tilt motion.

Upon determination in S164 that the instruction to terminate the head tilt motion has not been made, the motion assistance notification unit 21a executes the processing of S163.

Upon determination in S163 that the time measured by the timer is greater than or equal to the time that is the product of the specific unit time and the value of the variable t, the motion assistance notification unit 21a executes the processing of S137.

Upon determination in S161 that the time measured by the timer is greater than or equal to the specific time, the motion assistance notification unit 21a sets the write flag (S165).

Upon completion of the processing of S165, the motion assistance notification unit 21a writes the execution result of the current tilt and stop motion to the score determination information 20c (S166). Here, the motion assistance notification unit 21a calculates a mean value of all the tilts identified in S137 after the last execution of the processing of S135, identifies a detection area that is an area including the calculated mean value, and sets the identified detection area as an area in the execution result of the current tilt and stop motion. Further, the motion assistance notification unit 21a sets the time measured by the timer as duration of the stop of tilt change in the execution result of the current tilt and stop motion. That is, the motion assistance notification unit 21a identifies an actual execution detail of the head tilt motion on the basis of the tilt detected by the head tilt detection unit 16. Further, the motion assistance notification unit 21a calculates an integer obtained by rounding off a mean value of all the relaxation degrees determined in S139 after the last execution of the processing of S135 to the nearest whole number, and sets the calculated integer as the relaxation degree in the execution result of the current tilt and stop motion.

Upon completion of the processing of S166, the motion assistance notification unit 21a determines the recommended tilt of the stop of tilt change on the basis of the recommended tilt determination criteria 20e and the execution result of the head tilt motion indicated in the score determination information 20c (S167).

Upon completion of the processing of S167, the motion assistance notification unit 21a starts to make the tilt change instruction notification by, for example, at least one of display by the display unit 12, sound output by the sound output unit 13, or vibration output by the vibration output unit 14 (S168). The motion assistance notification unit 21a generates, as the tilt change instruction notification that starts to be made in S168, a notification for instructing to move the head of the user to the tilt determined in S167 and bring the head to rest. This allows the user who has received the tilt change instruction notification to move the head to the tilt indicated by the tilt change instruction notification and bring the head to rest.

Upon completion of the processing of S168, the motion assistance notification unit 21a starts to execute a notification of a state of the stop of tilt change (hereinafter, referred to as a “stop of tilt change state notification”) by, for example, at least one of display by the display unit 12, sound output by the sound output unit 13, or vibration output by the vibration output unit 14 (S169). This allows the user who has received the stop of tilt change state notification to recognize that the user has succeeded in bringing the head to rest.

Upon completion of the processing of S169, the motion assistance notification unit 21a starts to execute a notification of the relaxation degree determined in the last S139 by, for example, at least one of display by the display unit 12, sound output by the sound output unit 13, or vibration output by the vibration output unit 14 (S170). This allows the user to recognize his/her relaxation degree.

Upon completion of the processing of S170, the motion assistance notification unit 21a executes the processing of S162.

Upon determination in S140 that the write flag has not been cleared, the motion assistance notification unit 21a determines whether or not the head is at rest (S171). As a method for determining whether or not the head is at rest, various methods are applicable. The motion assistance notification unit 21a may determine that the head is at rest in a case where a deviation between all the tilts identified in S137 after the last execution of the processing of S135 falls within a specific range of the space shown in FIG. 2.

Upon determination in S171 that the head is at rest, the motion assistance notification unit 21a updates the execution result of the current tilt and stop motion in the score determination information 20c (S172). Here, the motion assistance notification unit 21a calculates a mean value of all the tilts identified in S137 after the last execution of the processing of S135, identifies a detection area that is an area including the calculated mean value, and sets the identified detection area as an area in the execution result of the current tilt and stop motion. Further, the motion assistance notification unit 21a sets the time measured by the timer as duration of the stop of tilt change in the execution result of the current tilt and stop motion. Further, the motion assistance notification unit 21a calculates an integer obtained by rounding off a mean value of all the relaxation degrees determined in S139 after the last execution of the processing of S135 to the nearest whole number, and sets the calculated integer as the relaxation degree in the execution result of the current tilt and stop motion.

Upon completion of the processing of S172, the motion assistance notification unit 21a executes the processing of S169.

Upon determination in S171 that the head is not at rest, the motion assistance notification unit 21a executes the processing of S133.

Upon determination in S134 or S164 that the instruction to terminate the head tilt motion has been made, the motion assistance notification unit 21a determines whether or not the output of the γ wave light by the light output unit 15 is in execution (S191).

Upon determination in S191 that the output of the γ wave light by the light output unit 15 is in execution, the motion assistance notification unit 21a terminates the output of the γ wave light by the light output unit 15 (S192).

Upon determination in S191 that the output of the γ wave light by the light output unit 15 is not in execution or upon completion of the processing of S192, the motion assistance notification unit 21a determines whether or not the output of the γ wave sound by the sound output unit 13 is in execution (S193).

Upon determination in S193 that the output of the γ wave sound by the sound output unit 13 is in execution, the motion assistance notification unit 21a terminates the output of the γ wave sound by the sound output unit 13 (S194).

Upon determination in S193 that the output of the γ wave sound by the sound output unit 13 is not in execution or upon completion of the processing of S194, the motion assistance notification unit 21a determines whether or not the execution result of the tilt and stop motion is included in the score determination information 20c (S195).

Upon determination in S195 that the score determination information 20c does not include the execution result of the tilt and stop motion, the motion assistance notification unit 21a terminates the operation shown in FIGS. 8 to 11.

Upon determination in S195 that the score determination information 20c includes the execution result of the tilt and stop motion, the motion assistance notification unit 21a executes score determination processing of determining the score of the current head tilt motion on the basis of the score determination information 20c and the score determination criteria 20d (S196).

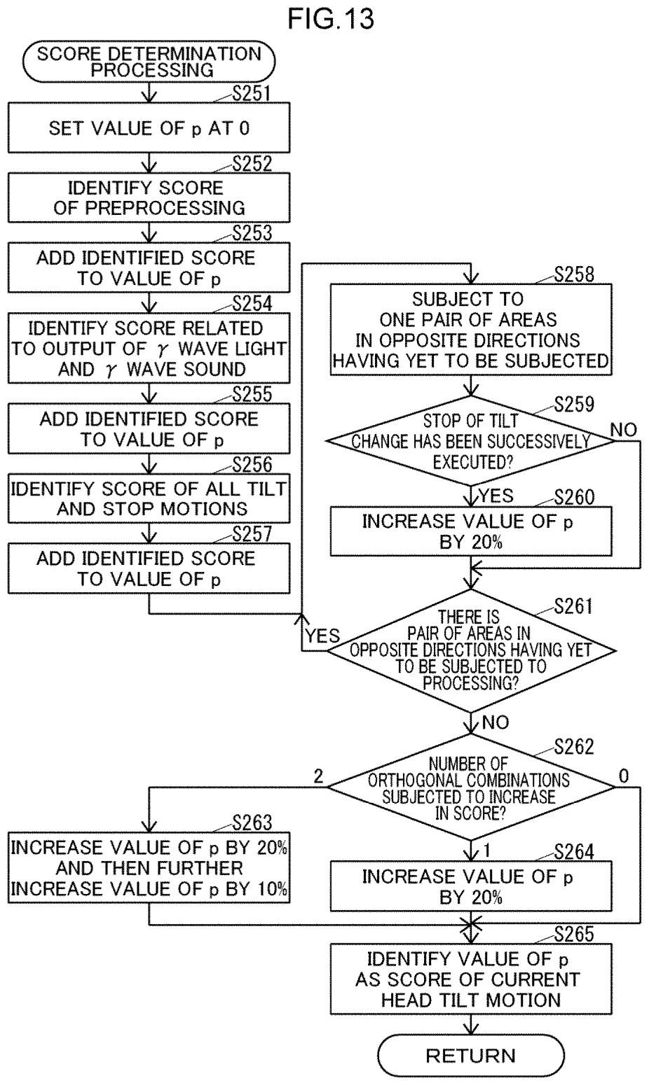

FIG. 13 is a flowchart of an example of the score determination processing shown in FIGS. 8 to 11.

Score determination processing shown in FIG. 13 is an example of the score determination processing in a case where the score determination criteria 20d are as shown in FIGS. 6A and 6B, and FIGS. 7A to 7D.

As shown in FIG. 13, the motion assistance notification unit 21a sets a value of a variable p at 0 (S251).

Upon completion of the processing of S251, the motion assistance notification unit 21a identifies the score of the preprocessing of the head tilt motion on the basis of the execution result of the preprocessing of the head tilt motion in the score determination information 20c and the criterion (see FIG. 6A) for the score of the preprocessing of the head tilt motion in the score determination criteria 20d (S252).

Upon completion of the processing of S252, the motion assistance notification unit 21a adds the score identified in S252 to the value of the variable p (S253).

Upon completion of the processing of S253, the motion assistance notification unit 21a identifies a score related to the output of the γ wave light and the output of the γ wave sound on the basis of the execution result of the output of the γ wave light and the output of the γ wave sound in the score determination information 20c and the criterion (see FIG. 6B) for the score of the output of the γ wave light and the output of the γ wave sound in the score determination criteria 20d (S254).

Upon completion of the processing of S254, the motion assistance notification unit 21a adds the score identified in S254 to the value of the variable p (S255).

Upon completion of the processing of S255, the motion assistance notification unit 21a identifies the scores of all the tilt and stop motions in the current head tilt motion on the basis of the execution result of the head tilt motion in the score determination information 20c and the criterion for the score of the tilt and stop motion (see FIG. 7A) in the score determination criteria 20d (S256). The motion assistance notification unit 21a, however, reduces the scores of the second and subsequent tilt and stop motions for the same area in the current head tilt motion by 50% (see FIG. 7B).

Upon completion of the processing of S256, the motion assistance notification unit 21a adds the scores identified in S256 to the value of the variable p (S257).

Upon completion of the processing of S257, the motion assistance notification unit 21a subjects to one pair of areas in opposite directions that has yet to be subjected to the processing of S258 in the current score determination processing (S258).

Upon completion of the processing of S258, the motion assistance notification unit 21a determines whether or not the stop of tilt change has been successively executed on each area in the pair of areas in opposite directions of the current target on the basis of the execution result of the head tilt motion in the score determination information 20c (S259).

Upon determination in S259 that the stop of tilt change has been successively executed on each area in the pair of areas in opposite directions of the current target, the motion assistance notification unit 21a increases the value of the variable p by 20% as shown in FIG. 7C (S260).

Upon determination in S259 that the stop of tilt change has not been successively executed on each area in the pair of areas in opposite directions of the current target or upon completion of the processing of S260, the motion assistance notification unit 21a determines whether or not there is a pair of areas in opposite directions that has yet to be subjected to the processing of S258 in the current score determination processing (S261).

Upon determination in S261 that there is a pair of areas in opposite directions that has yet to be subjected to the processing of S258 in the current score determination processing, the motion assistance notification unit 21a executes the processing of S258.

Upon determination in S261 that there is no pair of areas in opposite directions that has yet to be subjected to the processing of S258 in the current score determination processing, the motion assistance notification unit 21a determines the number of orthogonal combinations that are subjected to an increase in score in the current head tilt motion on the basis of the execution result of the head tilt motion in the score determination information 20c (S262).

Upon determination in S262 that the number of orthogonal combinations subjected to an increase in score in the current head tilt motion is two, the motion assistance notification unit 21a increases the value of the variable p by 20% and then further increases the value of the variable p by 10% as shown in FIG. 7D (S263).

Upon determination in S262 that the number of orthogonal combinations subjected to an increase in score in the current head tilt motion is one, the motion assistance notification unit 21a increases the value of the variable p by 20% as shown in FIG. 7D (S264).

Upon determination in S262 that the number of orthogonal combinations subjected to an increase in score in the current head tilt motion is zero or upon completion of the processing of S263 or S264, the motion assistance notification unit 21a identifies the value of the variable p as the score of the current head tilt motion (S265).

Upon completion of the processing of S265, the motion assistance notification unit 21a terminates the score determination processing shown in FIG. 13.

As shown in FIGS. 8 to 11, upon completion of the score determination processing in S196, the motion assistance notification unit 21a starts to make a notification of the score determined in S196 and the reason for the score, for example, by at least one of display by the display unit 12, sound output by the sound output unit 13, or vibration output by the vibration output unit 14 (S197). This allows the user to recognize the score of the current head tilt motion and the reason for the score. For example, as the reason for the score of the current head tilt motion, the motion assistance notification unit 21a may generate a breakdown of the score on the basis of the score determination information 20c and the score determination criteria 20d.

Upon completion of the processing of S197, the motion assistance notification unit 21a determines whether or not the reception of the input of the brain fatigue degree is set in the brain fatigue degree input setting of the setting information 20b (S198).

Upon determination in S198 that the reception of the input of the brain fatigue degree is set in the brain fatigue degree input setting of the setting information 20b, the motion assistance notification unit 21a displays an image for receiving the input of the brain fatigue degree (hereinafter, referred to as a “brain fatigue degree input reception image”) on the display unit 12 (S199). This allows the user to input, via the operation unit 11, the brain fatigue degree according to the brain fatigue degree input reception image displayed on the display unit 12.

Next, until the determination is made that the brain fatigue degree has been input via the operation unit 11, the motion assistance notification unit 21a repeats the determination of whether or not the brain fatigue degree has been input via the operation unit 11 (S200).

Upon determination in S198 that the reception of the input of the brain fatigue degree is not set in the brain fatigue degree input setting of the setting information 20b or upon determination in S200 that the brain fatigue degree has been input via the operation unit 11, that is, an input brain fatigue degree that is the brain fatigue degree input via the operation unit 11 has been acquired, the motion assistance notification unit 21a determines whether or not the execution of the brain fatigue degree measurement test is set in the test execution setting of the setting information 20b (S201).

Upon determination in S201 that the execution of the brain fatigue degree measurement test is set in the test execution setting of the setting information 20b, the motion assistance notification unit 21a executes the brain fatigue degree measurement test (S202).

Next, the motion assistance notification unit 21a makes a notification of a measured brain fatigue degree that is the brain fatigue degree measured by the brain fatigue degree measurement test executed in S202, by at least one of, for example, display by the display unit 12, sound output by the sound output unit 13, or vibration output by the vibration output unit 14 (S203).

Upon determination in S201 that the execution of the brain fatigue degree measurement test is not set in the test execution setting of the setting information 20b or upon completion of the processing of S203, the motion assistance notification unit 21a determines whether or not at least one of the input brain fatigue degree or the measured brain fatigue degree has been acquired (S204).

Upon determination in S204 that at least one of the input brain fatigue degree or the measured brain fatigue degree has been acquired, the motion assistance notification unit 21a determines whether or not the brain fatigue degree is greater than or equal to a specific value (S205). Here, in a case where both the input brain fatigue degree and the measured brain fatigue degree have been acquired, the motion assistance notification unit 21a may use a mean value of the input brain fatigue degree and the measured brain fatigue degree as the brain fatigue degree to be subjected to the determination in S205. In a case where only the input brain fatigue degree out of the input brain fatigue degree and the measured brain fatigue degree has been acquired, the motion assistance notification unit 21a uses the input brain fatigue degree as the brain fatigue degree to be subjected to the determination in S205. In a case where only the measured brain fatigue degree out of the input brain fatigue degree and the measured brain fatigue degree has been acquired, the motion assistance notification unit 21a uses the measured brain fatigue degree as the brain fatigue degree to be subjected to the determination in S205.

Upon determination in S205 that the brain fatigue degree is greater than or equal to the specific value, the motion assistance notification unit 21a sets the score determination criteria 20d to criteria different from default criteria (S206).

Upon determination in S205 that the brain fatigue degree is not greater than or equal to the specific value, the motion assistance notification unit 21a sets the score determination criteria 20d to the default criteria (S207).

Note that the score determination criteria 20d set in S206 make the score less likely to increase than the default criteria. Therefore, the motion assistance notification unit 21a sets the score determination criteria 20d in a case where the brain fatigue degree of the user is greater than or equal to the specific degree after the execution of the head tilt motion (YES in S205) to criteria under which the score is determined to be low as compared with the score determination criteria 20d in a case where the brain fatigue degree of the user is less than the specific degree after the execution of the head tilt motion (NO in S205) (S206).

Upon determination in S204 that neither the input brain fatigue degree nor the measured brain fatigue degree has been acquired or upon completion of the processing of S206 or S207, the motion assistance notification unit 21a terminates the operation shown in FIGS. 8 to 11.

Next, operation of the head tilt motion assistance system 10 in a case where an image representing a state where the cerebrospinal fluid moves in the brain in the gravity direction (hereinafter, referred to as a “cerebrospinal fluid movement image”) is displayed after the head tilt motion will be described.

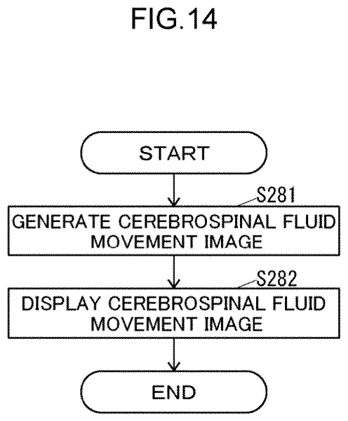

FIG. 14 is a flowchart of the operation of the head tilt motion assistance system 10 in a case where the cerebrospinal fluid movement image is displayed after the head tilt motion.

The user can instruct, via the operation unit 11, the head tilt motion assistance system 10 to display the cerebrospinal fluid movement image. Upon receipt of the instruction to display the cerebrospinal fluid movement image, the motion assistance notification unit 21a executes the operation shown in FIG. 14.

As shown in FIG. 14, the motion assistance notification unit 21a generates the cerebrospinal fluid movement image on the basis of the execution result of the head tilt motion indicated in the score determination information 20c (S281).

Next, the motion assistance notification unit 21a displays the cerebrospinal fluid movement image generated in S281 on the display unit 12 (S282), and terminates the operation shown in FIG. 14.

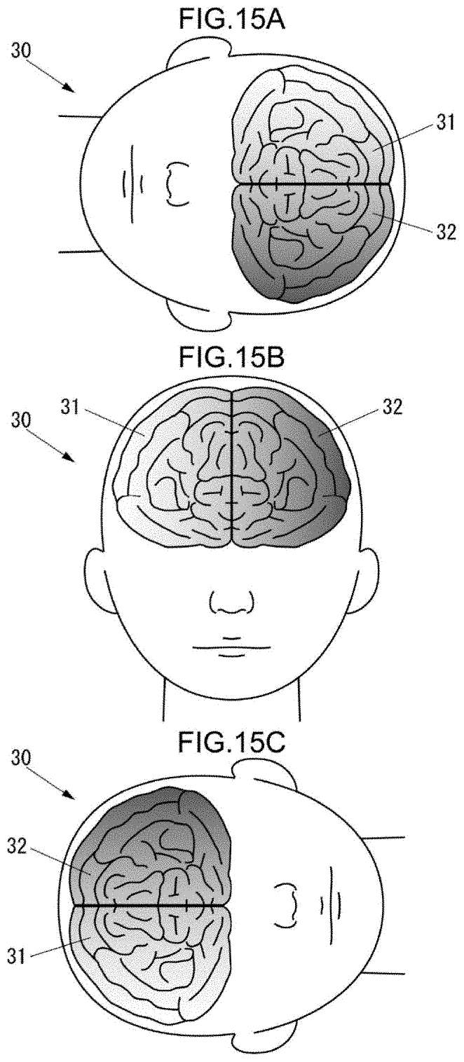

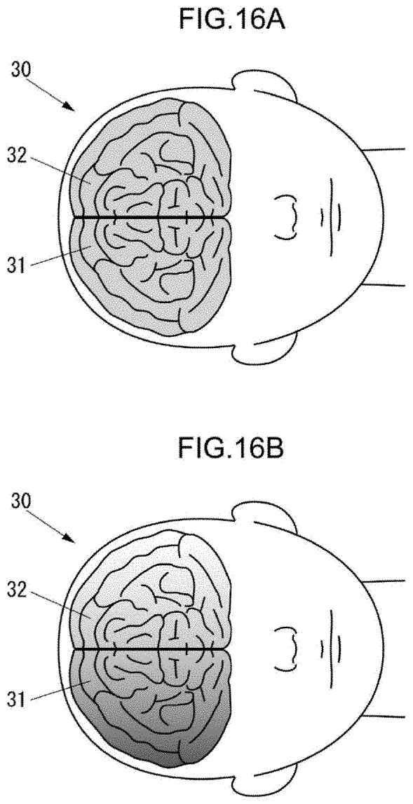

FIG. 15A is a diagram showing an example of a cerebrospinal fluid movement image 30 displayed on the display unit 12 at a specific timing in the head tilt motion. FIG. 15B is a diagram showing an example of the cerebrospinal fluid movement image 30 displayed on the display unit 12 at a specific timing after the timing shown in FIG. 15A. FIG. 15C is a diagram showing an example of the cerebrospinal fluid movement image 30 displayed on the display unit 12 at a specific timing after the timing shown in FIG. 15B. FIG. 16A is a diagram showing an example of the cerebrospinal fluid movement image 30 displayed on the display unit 12 at a specific timing after the timing shown in FIG. 15C. FIG. 16B is a diagram showing an example of the cerebrospinal fluid movement image 30 displayed on the display unit 12 at a specific timing after the timing shown in FIG. 16A.

The cerebrospinal fluid movement image 30 shown in FIGS. 15A to 15C and FIGS. 16A and 16B is a three-dimensional image including a brain 31 and cerebrospinal fluid 32. The motion assistance notification unit 21a tilts the brain 31 in the cerebrospinal fluid movement image 30 according to the direction and the angle of the tilt of the head of the user in the execution result of the head tilt motion indicated in the score determination information 20c in synchronization with the passage of time in the execution result of the head tilt motion indicated in the score determination information 20c. That is, the motion assistance notification unit 21a identifies the gravity direction with respect to the head on the basis of the tilt of the head of the user detected by the head tilt detection unit 16 in the head tilt motion, and changes the gravity direction with respect to the brain 31 in the cerebrospinal fluid movement image 30 according to the identified gravity direction with respect to the head. For example, according to the execution result of the head tilt motion indicated in the score determination information 20c, the brain 31 in the cerebrospinal fluid movement image 30 is changed from a state of being tilted to the left by 90° as shown in FIG. 15A to an upright state as shown in FIG. 15B, and then to a state of being tilted to the right by 90° as shown in FIG. 15C. Then, in a case where the brain 31 is in a state of being tilted to the right by 90°, the cerebrospinal fluid 32 in the cerebrospinal fluid movement image 30 gradually moves rightward in the brain 31 as shown in FIG. 16A due to gravity from a state of being unevenly present on the left side of the brain 31 as shown in FIG. 15C, and then becomes a state of being unevenly present on the right side of the brain 31 as shown in FIG. 16B. Here, the motion assistance notification unit 21a may change the ease of flow of the cerebrospinal fluid 32 in the cerebrospinal fluid movement image 30 in the brain 31 according to the execution result of the output of the γ wave light, the execution result of the output of the γ wave sound, and the relaxation degree in the score determination information 20c.

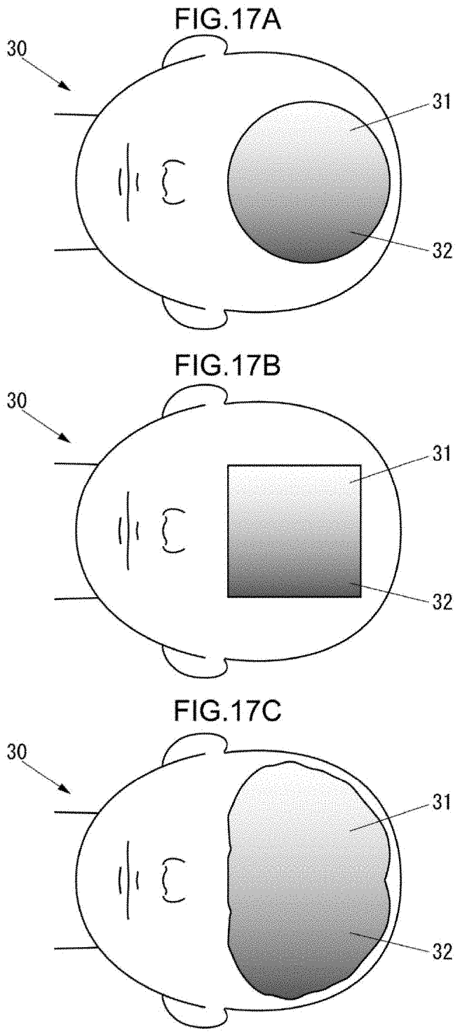

FIG. 17A is a diagram showing an example of the cerebrospinal fluid movement image 30 in a form of expression different from the form of expression shown in FIGS. 15A to 15C. FIG. 17B is a diagram showing an example of the cerebrospinal fluid movement image 30 in a form of expression different from the forms of expression shown in FIGS. 15A to 15C and FIG. 17A. FIG. 17C is a diagram showing an example of the cerebrospinal fluid movement image 30 in a form of expression different from the forms of expression shown in FIGS. 15A to 15C and FIGS. 17A and 17B.

The cerebrospinal fluid movement image 30 includes the brain 31 having a real shape in FIGS. 15A to 15D. In the cerebrospinal fluid movement image 30, the shape of the brain 31, however, may be simplified. For example, the shape of the brain 31 may be spherical as shown in FIG. 17A or cubic as shown in FIG. 17B.

The cerebrospinal fluid movement image 30 is a three-dimensional image in the above description. Alternatively, the cerebrospinal fluid movement image 30 may be a two-dimensional image as shown in FIG. 17C.

A case where the motion assistance notification unit 21a displays the cerebrospinal fluid movement image on the display unit 12 at the timing indicated by the user after the head tilt motion has been described above. Alternatively, the motion assistance notification unit 21a may display, in real time during the execution of the head tilt motion, the cerebrospinal fluid movement image of the head tilt motion on the display unit 12.

The flow of the cerebrospinal fluid shown in the cerebrospinal fluid movement image is preferably similar to the actual flow of the cerebrospinal fluid in the brain by the head tilt motion. That is, the cerebrospinal fluid movement image is preferably a simulation image of the flow of the cerebrospinal fluid in the brain by the head tilt motion. However, one of the purposes of the cerebrospinal fluid movement image is to give the user a motivation for the head tilt motion. For example, the user can execute the head tilt motion so as to cause the cerebrospinal fluid to flow in the brain as desired in the cerebrospinal fluid movement image. Therefore, the flow of the cerebrospinal fluid shown in the cerebrospinal fluid movement image may be completely different from the actual flow of the cerebrospinal fluid in the brain by the head tilt motion.

As described above, the head tilt motion assistance system 10 makes the motion assistance notification on the basis of the tilt of the head of the user detected by the head tilt detection unit 16 (S132, S168, S169, S197, and S282), so that it is possible to increase the possibility that the head tilt motion that is highly likely to cause the user to effectively recover from the fatigue of the brain is appropriately executed by the user. This allows the head tilt motion assistance system 10 to assist the user in recovering from the fatigue of the brain even with no brain function improving agent administered to the user.

Note that, for the head tilt motion assistance system 10, the head tilt motion may be executed with the brain function improving agent administered to the user.

The head tilt motion assistance system 10 determines the score of the head tilt motion on the basis of at least the actual execution detail of the head tilt motion and the score determination criteria 20d including the criterion according to the execution detail of the head tilt motion (S196), and notifies the determined score (S197), so that it is possible to increase the possibility that the head tilt motion is appropriately executed by the user.

When, in a case where the stop of tilt change is executed in a state where the tilt of the head of the user exists in the first area, the stop of tilt change executed immediately after the stop of tilt change executed in a state where the tilt of the head of the user exists in the first area is the stop of tilt change executed in a state where the tilt of the head of the user exists in the second area existing in the direction opposite to the first area, the head tilt motion assistance system 10 increases the score of the head tilt motion (see FIG. 7C), so that it is possible to increase the possibility that the head tilt motion that causes the cerebrospinal fluid to efficiently flow in the brain is executed by the user.

The head tilt motion assistance system 10 increases the score of the head tilt motion (see FIG. 7D) in a case where the stop of tilt change of the head of the user is executed in the head tilt motion in all of the state where the tilt of the head of the user exists in the first area, the state where the tilt of the head of the user exists in the second area existing in the direction opposite to the first area, the state where the tilt of the head of the user exists in the third area existing in the direction orthogonal to the second area, and the state where the tilt of the head of the user exists in the fourth area existing in the direction opposite to the third area, so that it is possible to increase the possibility that the head tilt motion that causes the cerebrospinal fluid to efficiently flow in the brain is executed by the user.

The head tilt motion assistance system 10 determines the score of the head tilt motion on the basis of at least the actual duration of the stop of tilt change of the head of the user in the head tilt motion and the score determination criteria 20d including the criterion (see FIG. 7A) according to the duration of the stop of tilt change of the head of the user in the head tilt motion (S196), so that it is possible to increase the possibility that the head tilt motion that causes the cerebrospinal fluid to efficiently flow in the brain is executed by the user.

The head tilt motion assistance system 10 determines the score of the head tilt motion on the basis of at least the actual relaxation degree of the user in the head tilt motion and the score determination criteria 20d including the criterion according to the relaxation degree of the user in the head tilt motion (S196), so that it is possible to increase the possibility that the head tilt motion is executed by the user in a state where the relaxation degree is high, that is, in a state where the user easily recovers from the fatigue of the brain.

The head tilt motion assistance system 10 determines the score of the head tilt motion on the basis of the tilt of the head of the user detected by the head tilt detection unit 16 in the head tilt motion and the relaxation degree of the user in the head tilt motion (S196), so that it is possible to increase the accuracy of the score of the head tilt motion.

The head tilt motion assistance system 10 determines the score of the head tilt motion on the basis of at least the actual execution detail of the preprocessing of the head tilt motion and the score determination criteria 20d including the criterion (see FIG. 6A) according to the execution detail of the preprocessing of the head tilt motion (S196), so that it is possible to increase the possibility that the preprocessing that causes the cerebrospinal fluid to efficiently flow in the brain is executed by the user.

The head tilt motion assistance system 10 makes a notification of the reason for the score of the head tilt motion (S197), so that it is possible to increase the possibility that the head tilt motion is appropriately executed by the user.

The head tilt motion assistance system 10 sets the score determination criteria 20d (S206) such that the score of the head tilt motion is determined to be low in a case where the brain fatigue degree of the user is low (YES in S205) after the execution of the head tilt motion, so that it is possible to increase the possibility that the head tilt motion that becomes high in score in a case where the brain fatigue degree of the user is low after the execution of the head tilt motion is positively executed by the user. This allows the head tilt motion assistance system 10 to increase the possibility that the head tilt motion is appropriately executed by the user.

The head tilt motion assistance system 10 makes a notification of the state of the stop of tilt change of the head of the user during the execution of the head tilt motion (S169), so that it is possible to increase the possibility that the head tilt motion that causes the cerebrospinal fluid to efficiently flow in the brain is executed by the user.

The head tilt motion assistance system 10 makes a notification of the relaxation degree of the user during the execution of the head tilt motion (S170), so that it is possible to cause the user to aim for a state where the relaxation degree is high, that is, a state where the user easily recovers from the fatigue of the brain during the execution of the head tilt motion.

The head tilt motion assistance system 10 makes a notification of the instruction to execute the preprocessing of the head tilt motion before executing the head tilt motion (S102), so that it is possible to increase the possibility that the preprocessing that causes the cerebrospinal fluid to efficiently flow in the brain is executed by the user.

The head tilt motion assistance system 10 determines the recommended tilt of the head of the user on the basis of at least the actual execution detail of the head tilt motion and the recommended tilt determination criteria 20e including the criterion according to the execution detail of the head tilt motion (S167), and makes a notification of the recommended tilt of the head of the user during the execution of the head tilt motion (S168), so that it is possible to increase the possibility that the head tilt motion that causes the user to effectively recover from the fatigue of the brain is appropriately executed by the user.

The head tilt motion assistance system 10 determines the tilt in the second area existing in the direction opposite to the first area as the recommended tilt of the head of the user (S167) when, in a case where the stop of tilt change is executed in a state where the tilt of the head of the user exists in the first area, the stop of tilt change executed immediately before the stop of tilt change executed in a state where the tilt of the head of the user exists in the first area is not the stop of tilt change executed in a state where the tilt of the head of the user exists in the second area, so that it is possible to increase the possibility that the head tilt motion that causes the cerebrospinal fluid to efficiently flow in the brain is executed by the user.

The head tilt motion assistance system 10 determines the tilt in the third area existing in the direction orthogonal to the second area existing in the direction opposite to the first area as the recommended tilt of the head of the user (S167) when, in a case where the stop of tilt change is executed in a state where the tilt of the head of the user exists in the first area, the stop of tilt change executed immediately before the stop of tilt change executed in a state where the tilt of the head of the user exists in the first area is the stop of tilt change executed in a state where the tilt of the head of the user exists in the second area, so that it is possible to increase the possibility that the head tilt motion that causes the cerebrospinal fluid to efficiently flow in the brain is executed by the user.

The head tilt motion assistance system 10 causes the light output unit 15 to output the γ wave light in the head tilt motion (S110). Therefore, the γ wave light that is output from the light output unit 15 comes within sight of the user during the execution of the head tilt motion, so that it is possible to effectively remove the brain fatigue stuff from the brain of the user. Further, the head tilt motion assistance system 10 causes the sound output unit 13 to output the γ wave sound in the head tilt motion (S114). Therefore, the γ wave sound that is output from the sound output unit 13 comes within hearing of the user during the execution of the head tilt motion, so that it is possible to effectively remove the brain fatigue stuff from the brain of the user. Note that the head tilt motion assistance system 10 can particularly effectively remove the brain fatigue stuff from the brain of the user by the γ wave light that is output from the light output unit 15 to come within sight of the user during the execution of the head tilt motion and the γ wave sound that is output from the sound output unit 13 to come within hearing of the user during the execution of the head tilt motion.

The head tilt motion assistance system 10 executes the brain fatigue degree measurement test after the execution of the head tilt motion (S202) and makes a notification of the brain fatigue degree measured by the brain fatigue degree measurement test (S203), so that it is possible to cause the user to grasp the effect of the head tilt motion. This allows the head tilt motion assistance system 10 to cause the user to aim to execute a head tilt motion that causes the user to effectively recover from the fatigue of the brain, such as a head tilt motion that causes the cerebrospinal fluid to efficiently flow in the brain.

In order to make a notification of the cerebrospinal fluid movement image in S282, the head tilt motion assistance system 10 identifies the gravity direction with respect to the head of the user on the basis of the tilt of the head detected by the head tilt detection unit 16 in the head tilt motion, and changes the gravity direction with respect to the brain in the cerebrospinal fluid movement image according to the identified gravity direction with respect to the head, so that it is possible to cause the user to aim to execute a head tilt motion that causes the cerebrospinal fluid to efficiently move in the brain in the cerebrospinal fluid movement image. This allows the head tilt motion assistance system 10 to cause the user to aim to execute a head tilt motion that causes the user to effectively recover from the fatigue of the brain.

As described above, upon determination in S205 that the brain fatigue degree is greater than or equal to the specific value, the motion assistance notification unit 21a sets the score determination criteria 20d to criteria different from the default criteria (S206). Alternatively, upon determination in S205 that the brain fatigue degree is greater than or equal to the specific value, the motion assistance notification unit 21a may make a notification for recommending the user to change the score determination criteria 20d, for example, by at least one of display by the display unit 12, sound output by the sound output unit 13, or vibration output by the vibration output unit 14 rather than automatically setting the score determination criteria 20d.

The motion assistance notification unit 21a may change various values indicated in the setting information 20b, the score determination information 20c, and the score determination criteria 20d according to an instruction from the user via the operation unit 11, for example.

According to the present embodiment, the head tilt motion assistance system 10 determines the relaxation degree on the basis of the brain waves measured by the brain wave measurement unit 17, the heart rate measured by the heart rate measurement unit 18, and the amount of mental perspiration measured by the mental perspiration measurement unit 19. Alternatively, the head tilt motion assistance system 10 may determine the relaxation degree on the basis of any one or two of the brain waves measured by the brain wave measurement unit 17, the heart rate measured by the heart rate measurement unit 18, and the amount of mental perspiration measured by the mental perspiration measurement unit 19. Further, the head tilt motion assistance system 10 may use information that is not the brain waves measured by the brain wave measurement unit 17, the heart rate measured by the heart rate measurement unit 18, or the amount of mental perspiration measured by the mental perspiration measurement unit 19 for the determination of the relaxation degree in addition to at least one of the brain waves measured by the brain wave measurement unit 17, the heart rate measured by the heart rate measurement unit 18, or the amount of mental perspiration measured by the mental perspiration measurement unit 19. The head tilt motion assistance system 10 may use information that is not the brain waves measured by the brain wave measurement unit 17, the heart rate measured by the heart rate measurement unit 18, or the amount of mental perspiration measured by the mental perspiration measurement unit 19 for the determination of the relaxation degree instead of at least one of the brain waves measured by the brain wave measurement unit 17, the heart rate measured by the heart rate measurement unit 18, or the amount of mental perspiration measured by the mental perspiration measurement unit 19.

As described above, the head tilt motion assistance system 10 includes the light output unit 15 as the γ wave light output unit separately from the display unit 12. Alternatively, in the head tilt motion assistance system 10, at least a part of the display unit 12 may serve as the γ wave light output unit. In a case where at least the part of the display unit 12 serves as the γ wave light output unit, the part of the display unit 12 serving as the γ wave light output unit may be, for example, a glasses-type display device.

### Second Embodiment

First, a configuration of a head tilt motion assistance system according to a second embodiment of the present invention will be described.

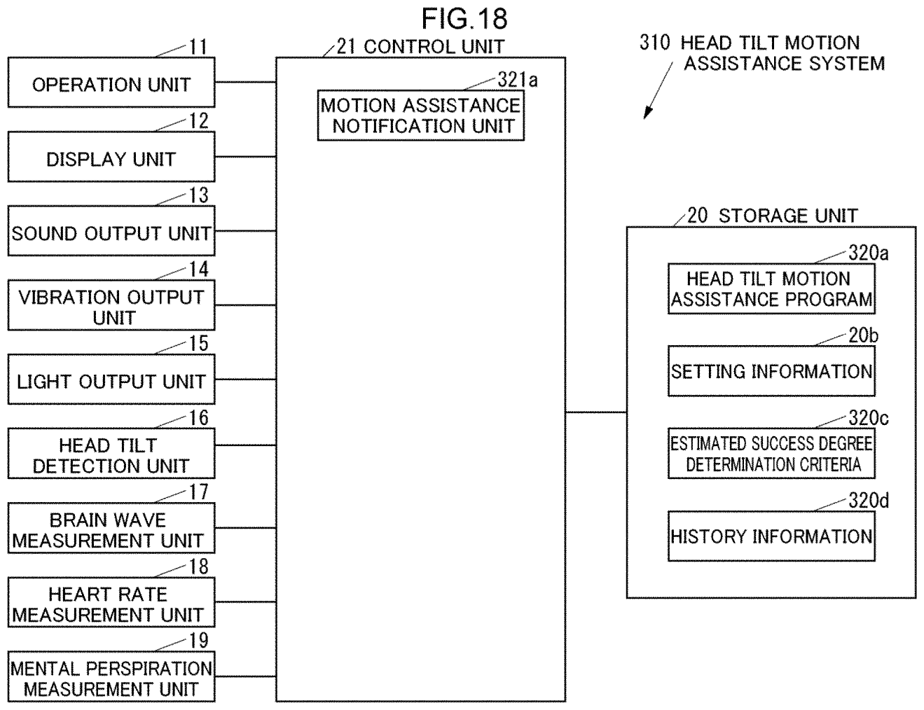

FIG. 18 is a block diagram of a head tilt motion assistance system 310 according to the present embodiment.

The configuration of the motion assistance system 310 shown in FIG. 18 is similar in configuration to the motion assistance system 10 (see FIG. 1.) except for the following configuration. Of components of the motion assistance system 310, components similar to the components of the motion assistance system 10 are denoted by the same reference numerals as the components of the motion assistance system 10, and no detailed description will be given of such components.

As illustrated in FIG. 18, the configuration of the motion assistance system 310 is similar to a configuration in which the motion assistance system 10 stores, in the storage unit 20, a head tilt motion assistance program 320a for assisting in the head tilt motion, estimated success degree determination criteria 320c indicating criteria (hereinafter referred to as “determination criteria”) for determination of a degree of success in estimation (hereinafter, referred to as an “estimated success degree”) of removal of the brain fatigue stuff from the brain by the cerebrospinal fluid, and history information 320d indicating a history related to the head tilt motion by the user (hereinafter, referred to as a “head tilt motion related history”) instead of the head tilt motion assistance program 20a (see FIG. 1), the score determination information 20c (see FIG. 1), the score determination criteria 20d (see FIG. 1), and the recommended tilt determination criteria 20e (see FIG. 1).

For example, the head tilt motion assistance program 320a may be installed on the head tilt motion assistance system 310 at a manufacturing stage of the head tilt motion assistance system 310, may be additionally installed on the head tilt motion assistance system 310 from an external storage medium such as a USB memory, or may be additionally installed on the head tilt motion assistance system 310 over a network.

The estimated success degree corresponds to the score of the head tilt motion. Therefore, the estimated success degree determination criteria 320c serve as the score determination criteria according to the present invention.

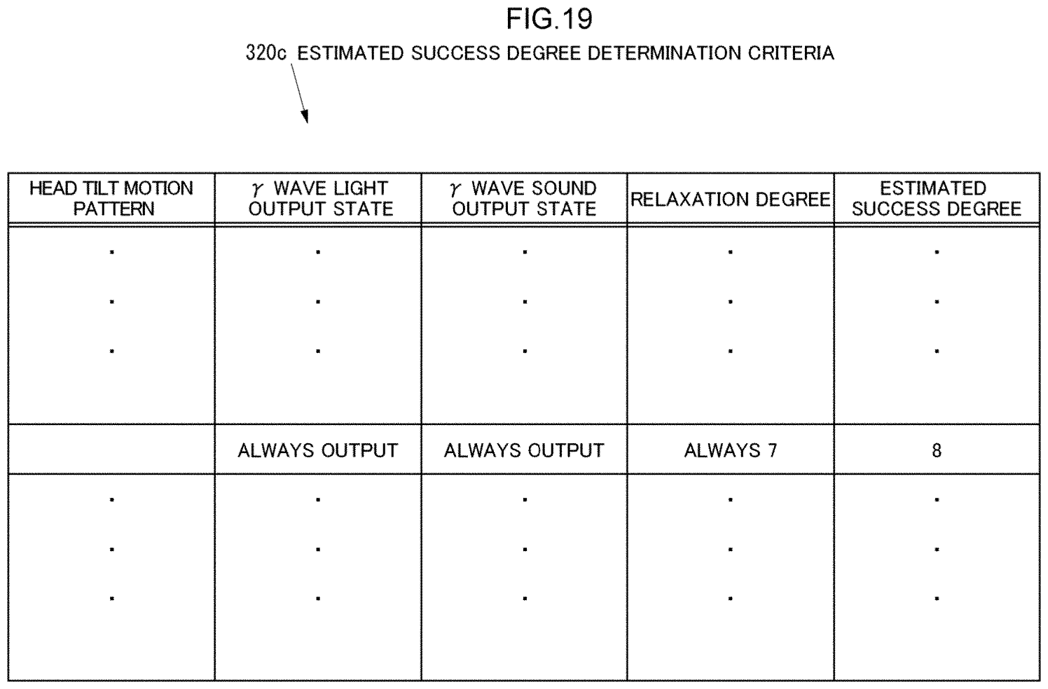

FIG. 19 is a diagram showing an example of the estimated success degree determination criteria 320c.

As shown in FIG. 19, the estimated success degree determination criteria 320c include a table including a head tilt motion pattern indicating a pattern of the head tilt motion, a state of the output of the γ wave light in the head tilt motion (hereinafter, referred to as a “γ wave light output state”), a state of the output of the γ wave sound in the head tilt motion (hereinafter, a “γ wave sound output state”), a relaxation degree of the user in the head tilt motion, and the estimated success degree for each combination of the head tilt motion pattern, the γ wave light output state, the γ wave sound output state, and the relaxation degree. The estimated success degree determination criteria 320c are criteria according to the head tilt motion pattern, that is, the execution detail of the head tilt motion.

The estimated success degree determination criteria 320c shown in FIG. 19 have a specific value for only one combination of the head tilt motion pattern, the γ wave light output state, the γ wave sound output state, and the relaxation degree included in the estimated success degree determination criteria 320c. In practice, however, the estimated success degree determination criteria 320c have specific values for all combinations of the head tilt motion pattern, the γ wave light output state, the γ wave sound output state, and the relaxation degree included in the estimated success degree determination criteria 320c.

The estimated success degree determination criteria 320c shown in FIG. 19 have no specific value of the head tilt motion pattern. In practice, however, the estimated success degree determination criteria 320c have a specific value of the head tilt motion pattern. The head tilt motion pattern in the estimated success degree determination criteria 320c is information indicating the time-series direction and angle of the tilt of the head.

An ideal head tilt motion pattern is, for example, a pattern in which making the head at rest for 20 seconds with the head tilted by 90° to the front relative to the upright state; making the head at rest for 20 seconds with the head tilted by 90° to the back relative to the upright state; making the head at rest or 20 seconds with the head tilted by 90° to the left relative to the upright state; making the head at rest for 20 seconds with the head tilted by 90° to the right relative to the upright state; making the head at rest for 20 seconds with the head tilted by 90° to the diagonally forward right relative to the upright state; making the head at rest for 20 seconds with the head tilted by 90° to the diagonally backward left relative to the upright state; making the head at rest for 20 seconds with the head tilted by 90° to the diagonally forward left relative to the upright state; and making the head at rest for 20 seconds with the head tilted by 90° to the diagonally backward right relative to the upright state are executed in sequence.

In the head tilt motion pattern, the order of the head tilt directions may not be the above-described order. Note that a case where the head is tilted to one of the front and the back, and then the head is tilted to the other allows the cerebrospinal fluid to efficiently flow in the brain by the head being tilted in the opposite directions, so that the estimated success degree in a case where the head is tilted to one of the front and the back, and then the head is tilted to the other may be set high in the estimated success degree determination criteria 320c as compared with the estimated success degree in a case where the head is tilted to one of the front and back, and then the head is not tilted to the other. Similarly, the estimated success degree in a case where the head is tilted to one of the left and the right, and then the head is tilted to the other may be set high in the estimated success degree determination criteria 320c as compared with the estimated success degree in a case where the head is tilted to one of the left and the right, and then the head is not tilted to the other. Further, the estimated success degree in a case where the head is tilted to one of the diagonally forward right and the diagonally backward left, and then the head is tilted to the other may also be set high in the estimated success degree determination criteria 320c as compared with the estimated success degree in a case where the head is tilted to one of the diagonally frontward right and the diagonally backward left, and then the head is not tilted to the other. Further, the estimated success degree in a case where the head is tilted to the diagonally forward left and the diagonally backward right, and then the head is tilted to the other may also be set high in the estimated success degree determination criteria 320c as compared with the estimated success degree in a case where the head is tilted to one of the diagonally frontward left and the diagonally backward right, and then the head is not tilted to the other. That is, the estimated success degree determination criteria 320c may be criteria for increasing the estimated success degree that is the score of the head tilt motion when, in a case where the stop of tilt change is executed in a state where the tilt of the head of the user is exists in the first area, the stop of tilt change executed immediately after the stop of tilt change executed in a state where the tilt of the head of the user is exists in the first area is the stop of tilt change executed in a state where the tilt of the head of the user exists in the second area existing in the direction opposite to the first area.

In the head tilt motion pattern, the angle of the tilt of the head need not necessarily be 90° described above.

In the head tilt motion pattern, the rest time after tilting the head need not necessarily be 20 seconds described above. For example, in the head tilt motion pattern, the rest time after tilting the head may be 10 seconds or 0 seconds. However, the brain gets fatigued due to the use of the brain to control muscles to continue to move the head, so that the rest time after tilting the head is preferably more than 0 seconds. Further, the cerebrospinal fluid flows slowly in the brain, so that the rest time after tilting the head is preferably more than 0 seconds.

The relaxation degree may be indicated on ten levels of 1 to 10, for example. The larger a numerical value of the relaxation degree, the more the brain is relaxed. It is known that the more the brain is relaxed, the more smoothly the cerebrospinal fluid flows in the brain. The relaxation degree in the estimated success degree determination criteria 320c may be indicated as varying in the head tilt motion.

The estimated success degree may be indicated on ten levels of 1 to 10, for example. The larger a numerical value of the estimated success degree, the higher the possibility that the brain fatigue stuff is efficiently removed from the brain by the cerebrospinal fluid. Of two records having the same combination of the head tilt motion pattern, the γ wave sound output state, and the relaxation degree, a record in which the γ wave light is output in the head tilt motion is set higher in the estimated success degree in the estimated success degree determination criteria 320c than a record in which the γ wave light is not output in the head tilt motion. Of two records having the same combination of the head tilt motion pattern, the γ wave light output state, and the relaxation degree, a record in which the γ wave sound is output in the head tilt motion is set higher in the estimated success degree in the estimated success degree determination criteria 320c than a record in which the γ wave sound is not output in the head tilt motion. Of two records having the same combination of the head tilt motion pattern, the γ wave light output state, and the γ wave sound output state, a record in which the relaxation degree is higher is set higher in the estimated success degree in the estimated success degree determination criteria 320c than a record in which the relaxation degree is lower.

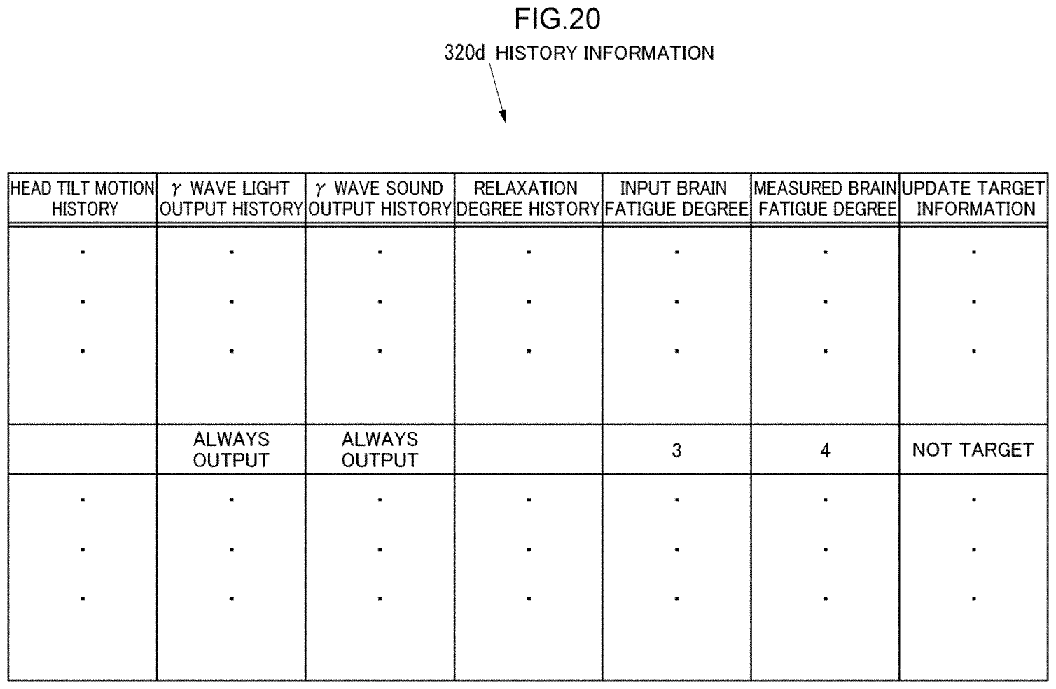

FIG. 20 is a diagram showing an example of the history information 320d.

As shown in FIG. 20, the history information 320d includes a table including, for each head tilt motion, a history of the head tilt motion (hereinafter, referred to as a “head tilt motion history”), a history of the γ wave light output state in the head tilt motion (hereinafter, referred to as a “γ wave light output history”), a history of the γ wave sound output state in the head tilt motion (hereinafter, referred to as a “γ wave sound output history”), a history of the relaxation degree in the head tilt motion (hereinafter, referred to as a “relaxation degree history”), an input brain fatigue degree that is the brain fatigue degree input by the user after the head tilt motion, a measured brain fatigue degree that is the brain fatigue degree measured by the brain fatigue degree measurement test after the head tilt motion, and update target information indicating whether or not it is to be updated at the time of updating the estimated success degree determination criteria 320c.

The history information 320d shown in FIG. 20 has a specific value for only one head tilt motion included in the history information 320d. In practice, however, the history information 320d has specific values for all the head tilt motions included in the history information 320d.

The history information 320d shown in FIG. 20 has no specific value of the head tilt motion history. In practice, however, the history information 320d has a specific value of the head tilt motion history. The head tilt motion history in the history information 320d is information indicating the time-series direction and angle of the tilt of the head of the user detected by head tilt detection unit 16.

The history information 320d shown in FIG. 20 has no specific value of the relaxation degree history. In practice, however, the history information 320d has a specific value of the relaxation degree history. The relaxation degree history in the history information 320d is information indicating the time-series relaxation degree determined on the basis of the brain waves measured by the brain wave measurement unit 17, the heart rate measured by the heart rate measurement unit 18, and the amount of mental perspiration measured by the mental perspiration measurement unit 19.

The input brain fatigue degree may be indicated on ten levels of 1 to 10, for example. The larger a numerical value of the input brain fatigue degree, the more the brain is fatigued.

The measured brain fatigue degree may be indicated on ten levels of 1 to 10, for example. The larger a numerical value of the measured brain fatigue degree, the more the brain is fatigued.

The control unit 21 shown in FIG. 18 executes the head tilt motion assistance program 320a to implement a motion assistance notification unit 321a that makes a motion assistance notification that is a notification for assisting in the head tilt motion.

Next, the operation of the head tilt motion assistance system 310 will be described.

First, the operation of the head tilt motion assistance system 310 for updating the setting information 20b will be described.

The operation of the head tilt motion assistance system 310 for updating the setting information 20b is similar to the operation of the head tilt motion assistance system 10 for updating the setting information 20b.

Next, the operation of the head tilt motion assistance system 310 upon receipt of an instruction to start the head tilt motion will be described.

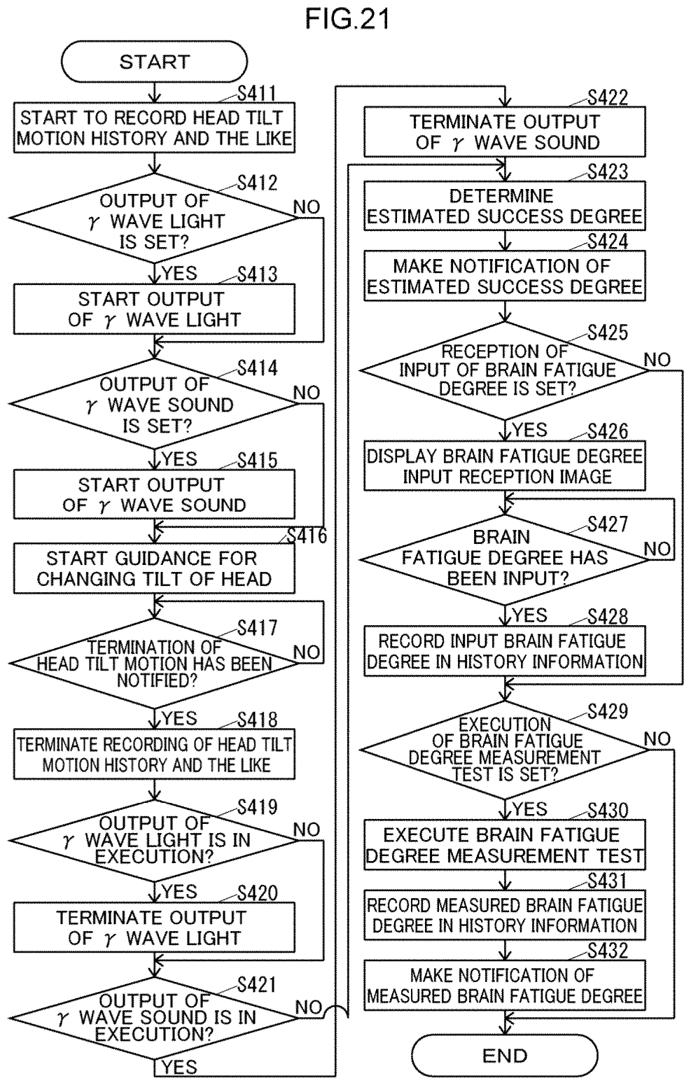

FIG. 21 is a flowchart of the operation of the head tilt motion assistance system 310 upon receipt of the instruction to start the head tilt motion.

In order to start the head tilt motion, the user can instruct, via the operation unit 11, the head tilt motion assistance system 310 to start the head tilt motion. Upon receipt of the instruction to start the head tilt motion, the motion assistance notification unit 321a executes the operation shown in FIG. 21.

As shown in FIG. 21, the motion assistance notification unit 321a starts to record the head tilt motion history, the γ wave light output history, the γ wave sound output history, and the relaxation degree history for the head tilt motion currently in execution (hereinafter, referred to as a “current head tilt motion”) in the history information 320d (S411). That is, the motion assistance notification unit 321a identifies an actual execution detail of the head tilt motion on the basis of the tilt detected by the head tilt detection unit 16. The motion assistance notification unit 321a determines the relaxation degree in a similar manner to the motion assistance notification unit 21a according to the first embodiment.

Upon completion of the processing of S411, the motion assistance notification unit 321a determines whether or not the output of the γ wave light is set in the γ wave light output setting of the setting information 20b (S412).

Upon determination in S412 that the output of the γ wave light is set in the γ wave light output setting of the setting information 20b, the motion assistance notification unit 321a starts the output of the γ wave light by the light output unit 15 (S413). This brings the γ wave light output by the light output unit 15 within sight of the user.

Upon determination in S412 that the output of the γ wave light is not set in the γ wave light output setting of the setting information 20b or upon completion of the processing of S413, the motion assistance notification unit 321a determines whether or not the output the γ wave sound is set in the γ wave sound output setting of the setting information 20b (S414).

Upon determination in S414 that the output of the γ wave sound is set in the γ wave sound output setting of the setting information 20b, the motion assistance notification unit 321a starts the output of the γ wave sound by the sound output unit 13 (S415). This brings the γ wave sound output by the sound output unit 13 within hearing of the user.

Upon determination in S414 that the output of the γ wave sound is not set in the γ wave sound output setting of the setting information 20b or upon completion of the processing of S415, the motion assistance notification unit 321a starts guidance for changing the tilt of the head according to a head tilt motion pattern having the highest estimated success degree among the head inclination motion patterns associated, in the estimated success degree determination criteria 320c, with the combination of the γ wave light output state set in the γ wave light output setting of the setting information 20b and the γ wave sound output state set in the γ wave sound output setting of the setting information 20b by, for example, at least one of sound output by the sound output unit 13 or vibration output by the vibration output unit 14 (S416). This allows the user to execute the current head tilt motion in accordance with the guidance executed by the motion assistance notification unit 321a.

In order to terminate the current head tilt motion, the user can notify, via the operation unit 11, the head tilt motion assistance system 310 of the termination of the head tilt motion. Upon completion of the processing of S416, the motion assistance notification unit 321a repeats the determination of whether or not the termination of the head tilt motion has been notified until the determination is made that the termination of the head tilt motion has been notified (S417).

Upon determination in S417 that the termination of the head tilt motion has been notified, the motion assistance notification unit 321a terminates the recording of the head tilt motion history, the γ wave light output history, the γ wave sound output history, and the relaxation degree history for the current head tilt motion in the history information 320d (S418).

Next, the motion assistance notification unit 321a determines whether or not the output of the γ wave light by the light output unit 15 is in execution (S419).

Upon determination in S419 that the output of the γ wave light by the light output unit 15 is in execution, the motion assistance notification unit 321a terminates the output of the γ wave light by the light output unit 15 (S420).

Upon determination in S419 that the output of the γ wave light by the light output unit 15 is not in execution or upon completion of the processing of S420, the motion assistance notification unit 321a determines whether or not the output of the γ wave sound by the sound output unit 13 is in execution (S421).

Upon determination in S421 that the output of the γ wave sound by the sound output unit 13 is in execution, the motion assistance notification unit 321a terminates the output of the γ wave sound by the sound output unit 13 (S422).

Upon determination in S421 that the output of the γ wave sound by the sound output unit 13 is not in execution or upon completion of the processing of S422, the motion assistance notification unit 321a determines the estimated success degree for the current head tilt motion on the basis of the estimated success degree determination criteria 320c, the head tilt motion history, the γ wave light output history, the γ wave sound output history, and the relaxation degree history for the current head tilt motion (S423). For example, the motion assistance notification unit 321a may make the determination in S423 using a machine learning model built using the estimated success degree determination criteria 320c as training data.

Next, the motion assistance notification unit 321a makes a notification of the estimated success degree determined in S423 by, for example, at least one of display by the display unit 12, sound output by the sound output unit 13, or vibration output by the vibration output unit 14 (S424).

Next, the motion assistance notification unit 321a determines whether or not the reception of the input of the brain fatigue degree is set in the brain fatigue degree input setting of the setting information 20b (S425).

Upon determination in S425 that the reception of the input of the brain fatigue degree is set in the brain fatigue degree input setting of the setting information 20b, the motion assistance notification unit 321a displays the brain fatigue degree input reception image on the display unit 12 (S426). This allows the user to input, via the operation unit 11, the brain fatigue degree according to the brain fatigue degree input reception image displayed on the display unit 12.

Next, until the determination is made that the brain fatigue degree has been input via the operation unit 11, the motion assistance notification unit 321a repeats the determination of whether or not the brain fatigue degree has been input via the operation unit 11 (S427).

Upon determination in S427 that the brain fatigue degree has been input via the operation unit 11, the motion assistance notification unit 321a records the brain fatigue degree input via the operation unit 11, that is, the input brain fatigue degree, in the history information 320d (S428).

Upon determination in S425 that the reception of the input of the brain fatigue degree is not set in the brain fatigue degree input setting of the setting information 20b or upon completion of the processing of S428, the motion assistance notification unit 321a determines whether or not the execution of the brain fatigue degree measurement test is set in the test execution setting of the setting information 20b (S429).

Upon determination in S429 that the execution of the brain fatigue degree measurement test is set in the test execution setting of the setting information 20b, the motion assistance notification unit 321a executes the brain fatigue degree measurement test (S430).

Next, the motion assistance notification unit 321a records the brain fatigue degree measured by the brain fatigue degree measurement test executed in S430, that is, the measured brain fatigue degree, in the history information 320d (S431).

Next, the motion assistance notification unit 321a makes a notification of the measured brain fatigue degree measured by the brain fatigue degree measurement test executed in S430, by at least one of, for example, display by the display unit 12, sound output by the sound output unit 13, or vibration output by the vibration output unit 14 (S432).

Upon determination in S429 that the execution of the brain fatigue degree measurement test is not set in the test execution setting of the setting information 20b or upon completion of the processing of S432, the motion assistance notification unit 321a terminates the operation shown in FIG. 21.

As described above, the motion assistance notification unit 321a outputs the γ wave light without interruption during the execution of the head tilt motion. Alternatively, the motion assistance notification unit 321a may output the γ wave light only when the head has a specific tilt during the execution of the head tilt motion, or need not output the γ wave light at all during the execution of the head tilt motion. In a case where the γ wave light is not output at all during the execution of the head tilt motion, the head tilt motion assistance system 310 need not include the light output unit 15.

As described above, the motion assistance notification unit 321a outputs the γ wave sound without interruption during the execution of the head tilt motion. Alternatively, the motion assistance notification unit 321a may output the γ wave sound only when the head has a specific tilt during the execution of the head tilt motion, or need not output the γ wave sound at all during the execution of the head tilt motion.

As described above, the motion assistance notification unit 321a executes the guidance for changing the tilt of the head by, for example, at least one of sound output by the sound output unit 13 or vibration output by the vibration output unit 14. The motion assistance notification unit 321a may execute the guidance for changing the tilt of the head by display of an image by the display unit 12 instead of at least one of sound output by the sound output unit 13 or vibration output by the vibration output unit 14. The motion assistance notification unit 321a may execute the guidance for changing the tilt of the head by display of an image by the display unit 12 in addition to at least one of sound output by the sound output unit 13 or vibration output by the vibration output unit 14.

The motion assistance notification unit 321a may make the notification according to the relaxation degree of the user in real time during the execution of the head tilt motion by, for example, at least one of display by the display unit 12, sound output by the sound output unit 13, or vibration output by the vibration output unit 14. For example, in a case where the relaxation degree of the user is a specific high degree during the execution of the head tilt motion, the motion assistance notification unit 321a may make a notification that the relaxation degree of the user is the specific high degree by, for example, at least one of display by the display unit 12, sound output by the sound output unit 13, or vibration output by the vibration output unit 14.

Next, the operation of the head tilt motion assistance system 310 in a case where the cerebrospinal fluid movement image is displayed after the head tilt motion will be described.

The operation of the head tilt motion assistance system 310 in a case where the cerebrospinal fluid movement image is displayed after the head tilt motion is similar to the operation of the head tilt motion assistance system 10 in a case where the cerebrospinal fluid movement image is displayed after the head tilt motion except for the following content.

In order for the user to instruct the head tilt motion assistance system 310 to display the cerebrospinal fluid movement image via the operation unit 11, the user designates, via the operation unit 11, a head tilt motion to be the target of the cerebrospinal fluid movement image among the head tilt motions indicated by the history information 320d. Then, the motion assistance notification unit 321a generates the cerebrospinal fluid movement image on the basis of the head tilt motion history for the head tilt motion designate by the user (hereinafter, referred to as a “designated head tilt motion”) among the head tilt motions indicated by the history information 320d. The motion assistance notification unit 321a tilts the brain in the cerebrospinal fluid movement image according to the direction and the angle of the tilt of the head of the user in the head tilt motion history for the designated head tilt motion in synchronization with the passage of time in the head tilt motion history for the designated head tilt motion. Here, the motion assistance notification unit 321a may change the ease of flow of the cerebrospinal fluid in the brain in the cerebrospinal fluid movement image according to the γ wave light output history, the γ wave sound output history, and the relaxation degree history for the designated head tilt motion in the history information 320d.

The case where the motion assistance notification unit 321a displays the cerebrospinal fluid movement image on the display unit 12 at the timing indicated by the user after the head tilt motion has been described above. Alternatively, the motion assistance notification unit 321a may display the cerebrospinal fluid movement image of the head tilt motion on the display unit 12 in real time during the execution of the head tilt motion.

Next, the operation of the head tilt motion assistance system 310 for updating the estimated success degree determination criteria 320c will be described.

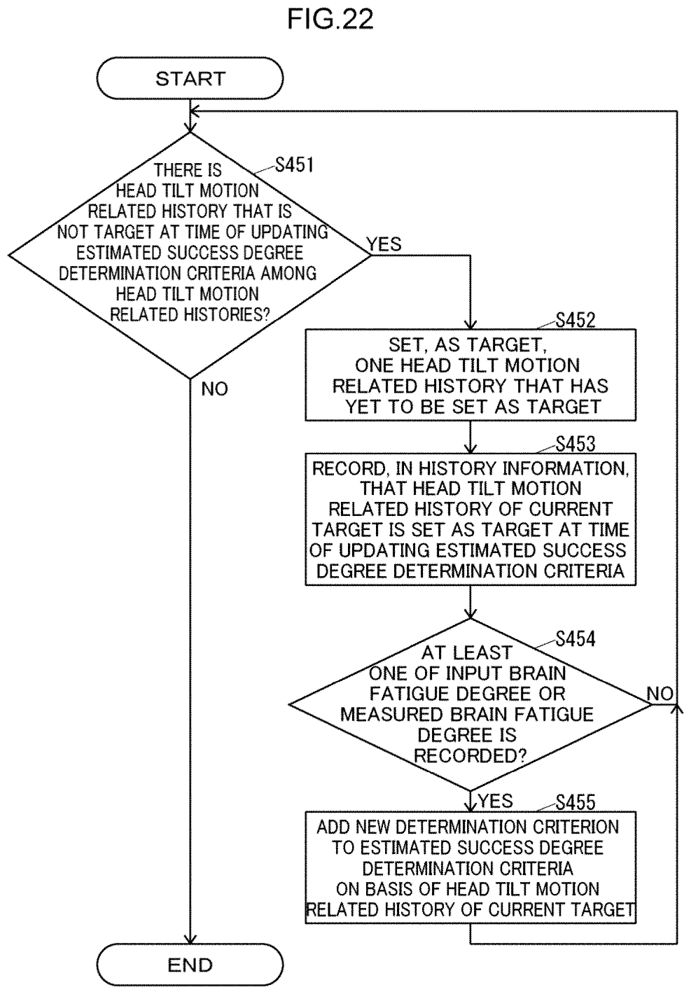

FIG. 22 is a flowchart of the operation of the head tilt motion assistance system 310 for updating the estimated success degree determination criteria 320c.

The motion assistance notification unit 321a executes the operation shown in FIG. 22 at a specific timing. The timing at which the operation shown in FIG. 22 is executed by the motion assistance notification unit 321a may be, for example, a periodic timing or a timing at which the user instructs the head tilt motion assistance system 310 to update the estimated success degree determination criteria 320c via the operation unit 11.

As shown in FIG. 22, the motion assistance notification unit 321a determines whether or not there is a head tilt motion related history that is not a target at the time of updating the estimated success degree determination criteria 320c among the head tilt motion related histories indicated in the history information 320d on the basis of the update target information in the history information 320d (S451).

Upon determination in S451 that there is a head tilt motion related history that is not a target at the time of updating the estimated success degree determination criteria 320c among the head tilt motion related histories indicated in the history information 320d, the motion assistance notification unit 321a sets, as a target, one head tilt motion related history that has yet to be set as a target among the head tilt motion related histories that have yet to be set as a target at the time of updating the estimated success degree determination criteria 320c indicated in the history information 320d (S452).

Next, the motion assistance notification unit 321a records, in the update target information in the history information 320d, that the head tilt motion related history of the current target has been set as a target at the time of updating the estimated success degree determination criteria 320c (S453).

Next, the motion assistance notification unit 321a determines whether or not at least one of the input brain fatigue degree or the measured brain fatigue degree is recorded in the history information 320d for the head tilt motion related history of the current target (S454).

Upon determination in S454 that at least one of the input brain fatigue degree or the measured brain fatigue degree is recorded in the history information 320d, the motion assistance notification unit 321a adds a new determination criterion to the estimated success degree determination criteria 320c on the basis of the head tilt motion related history of the current target (S455).

In the processing of S455, the motion assistance notification unit 321a sets the head tilt motion history, the γ wave light output history, the γ wave sound output history, and the relaxation degree history in the head tilt motion related history of the current target as the head tilt motion pattern, the γ wave light output state, the γ wave sound output state, and the relaxation degree in the estimated success degree determination criteria 320c, respectively.

In the processing of S455, the motion assistance notification unit 321a determines the estimated success degree in the estimated success degree determination criteria 320c according to a specific rule on the basis of at least one of the input brain fatigue degree or the measured brain fatigue degree in the head tilt motion related history of the current target. In a case where only the input brain fatigue degree out of the input brain fatigue degree and the measured brain fatigue degree is included in the head tilt motion related history of the current target, when the estimated success degree and the input brain fatigue degree are each indicated on ten levels of 1 to 10, for example, the motion assistance notification unit 321a may set a numerical value obtained by subtracting a numerical value of the input brain fatigue degree from 11 as the estimated success degree. In a case where only the measured brain fatigue degree out of the input brain fatigue degree and the measured brain fatigue degree is included in the head tilt motion related history of the current target, when the estimated success degree and the measured brain fatigue degree are each indicated on ten levels of 1 to 10, for example, the motion assistance notification unit 321a may set a numerical value obtained by subtracting a numerical value of the measured brain fatigue degree from 11 as the estimated success degree. In a case where the input brain fatigue degree and the measured brain fatigue degree are included in the head tilt motion related history of the current target, when the estimated success degree, the input brain fatigue degree, and the measured brain fatigue degree are each indicated on ten levels of 1 to 10, for example, the motion assistance notification unit 321a may set a numerical value obtained by dropping the fractional portion of a value obtained by subtracting a mean value of the input brain fatigue degree and the measured brain fatigue degree from 11 as the estimated success degree.

Upon determination in S454 that neither the input brain fatigue degree nor the measured brain fatigue degree is recorded in the history information 320d or upon completion of the processing of S455, the motion assistance notification unit 321a executes the processing of S451.

Upon determination in S451 that there is no head tilt motion related history that is not a target at the time of updating the estimated success degree determination criteria 320c among the head tilt motion related histories indicated in the history information 320d, the motion assistance notification unit 321a terminates the operation shown in FIG. 22.

As described above, the head tilt motion assistance system 310 makes the motion assistance notification on the basis of the tilt of the head of the user detected by the head tilt detection unit 16 (S424), so that it is possible to increase the possibility that the head tilt motion that is highly likely to cause the user to effectively recover from the fatigue of the brain is appropriately executed by the user. This allows the head tilt motion assistance system 310 to assist the user in recovering from the fatigue of the brain even with no brain function improving agent administered to the user.

Note that, for the head tilt motion assistance system 310, the head tilt motion may be executed with the brain function improving agent administered to the user.

The head tilt motion assistance system 310 determines the estimated success degree as the score of the head tilt motion on the basis of at least the actual execution detail of the head tilt motion and the estimated success degree determination criteria 320c including the criterion according to the execution detail of the head tilt motion (S423), and makes a notification of the determined estimated success degree (S424), so that it is possible to increase the possibility that the head tilt motion is appropriately executed by the user.

The head tilt motion assistance system 310 makes a notification of the estimated success degree that is a degree of success in estimation of removal of the brain fatigue stuff from the brain by the cerebrospinal fluid (S424), so that it is possible for the user to grasp, using the estimated success degree, how efficiently the brain fatigue stuff has been removed from the brain by the cerebrospinal fluid by the head tilt motion. This allows the head tilt motion assistance system 310 to cause the user to aim to execute a head tilt motion that causes the cerebrospinal fluid to efficiently flow in the brain.

When, in a case where the stop of tilt change is executed in a state where the tilt of the head of the user exists in the first area, the stop of tilt change executed immediately after the stop of tilt change executed in a state where the tilt of the head of the user exists in the first area is the stop of tilt change executed in a state where the tilt of the head of the user exists in the second area existing in the direction opposite to the first area, the head tilt motion assistance system 310 increases the estimated success degree that is the score of the head tilt motion, so that it is possible to increase the possibility that the head tilt motion that causes the cerebrospinal fluid to efficiently flow in the brain is executed by the user.

The head tilt motion assistance system 310 determines the estimated success degree on the basis of at least the actual relaxation degree of the user in the head tilt motion and the estimated success degree determination criteria 320c including the criterion according to the relaxation degree of the user in the head tilt motion (S423), so that it is possible to increase the possibility that the head tilt motion is executed by the user in a state where the relaxation degree is high, that is, in a state where the user easily recovers from the fatigue of the brain.

The head tilt motion assistance system 310 determines the estimated success degree on the basis of the tilt of the head of the user detected by the head tilt detection unit 16 in the head tilt motion and the relaxation degree of the user in the head tilt motion (S423), so that it is possible to increase the accuracy of the estimated success degree.

The head tilt motion assistance system 310 causes the light output unit 15 to output the γ wave light in the head tilt motion (S413). Therefore, the γ wave light that is output from the light output unit 15 comes within sight of the user during the execution of the head tilt motion, so that it is possible to effectively remove the brain fatigue stuff from the brain of the user. Further, the head tilt motion assistance system 310 causes the sound output unit 13 to output the γ wave sound in the head tilt motion (S415). Therefore, the γ wave sound that is output from the sound output unit 13 comes within hearing of the user during the execution of the head tilt motion, so that it is possible to effectively remove the brain fatigue stuff from the brain of the user. Note that the head tilt motion assistance system 310 can particularly effectively remove the brain fatigue stuff from the brain of the user by the γ wave light that is output from the light output unit 15 to come within sight of the user during the execution of the head tilt motion and the γ wave sound that is output from the sound output unit 13 to come within hearing of the user during the execution of the head tilt motion.

The head tilt motion assistance system 310 executes the brain fatigue degree measurement test after the execution of the head tilt motion (S430) and makes a notification of the brain fatigue degree measured by the brain fatigue degree measurement test (S432), so that it is possible to cause the user to grasp the effect of the head tilt motion. This allows the head tilt motion assistance system 310 to cause the user to aim to execute a head tilt motion that causes the user to effectively recover from the fatigue of the brain, such as a head tilt motion that causes the cerebrospinal fluid to efficiently flow in the brain.

The head tilt motion assistance system 310 executes the guidance for changing the tilt of the head of the user in the head tilt motion (S416), so that it is possible to cause the user to execute the head tilt motion that causes the cerebrospinal fluid to efficiently flow in the brain. In particular, the head tilt motion assistance system 310 can cause the user to execute the head tilt motion that causes the cerebrospinal fluid to efficiently flow in the brain in a case where guidance for tilting the head of the user in a direction opposite to a specific direction after tilting the head of the user in the specific direction.

The head tilt motion assistance system 310 updates the estimated success degree determination criteria on the basis of the history of the head tilt motion executed by the user (S455), so that the estimated success degree determination criteria can be made suitable for the user. This allows the head tilt motion assistance system 310 to increase the accuracy of the estimated success degree.

The cerebrospinal fluid smoothly flowing in the brain as the brain is relaxed. The head tilt motion assistance system 310 makes a notification according to the relaxation degree of the user in the head tilt motion, so that it is possible to cause the user to aim for a higher relaxation degree such that the cerebrospinal fluid efficiently flows in the brain during the execution of the head tilt motion.

According to the present embodiment, the head tilt motion assistance system 310 makes a notification of the estimated success degree and the display of the cerebrospinal fluid movement image as the notification for assisting in the head tilt motion on the basis of the direction and the angle of the tilt of the head of the user detected by the head tilt detection unit 16 in the head tilt motion. Alternatively, the head tilt motion assistance system 310 may makes a notification that is neither the notification of the estimated success degree nor the display of the cerebrospinal fluid movement image as the notification for assisting in the head tilt motion on the basis of the direction and the angle of the tilt of the head of the user detected by the head tilt detection unit 16 in the head tilt motion.

What is claimed is:

1. A head tilt motion assistance system comprising:
a head tilt detection unit that detects a tilt of a head of a user; and
a motion assistance notification unit that makes a motion assistance notification that is a notification for assisting in a head tilt motion that is a motion in which the user tilts the head, wherein
the motion assistance notification unit makes the motion assistance notification on a basis of the tilt detected by the head tilt detection unit.

2. The head tilt motion assistance system according to claim 1, wherein
the motion assistance notification includes a notification of a score of the head tilt motion,
the motion assistance notification unit identifies an actual execution detail of the head tilt motion on a basis of the tilt detected by the head tilt detection unit,
the motion assistance notification unit determines the score on a basis of on at least score determination criteria that are criteria for determination of the score and the actual execution detail of the head tilt motion, and
the score determination criteria include a criterion according to an execution detail of the head tilt motion.

3. The head tilt motion assistance system according to claim 2, wherein
the score determination criteria include a criterion for increasing the score when, in a case where a stop of tilt change that is a stop of change in the tilt in the head tilt motion is executed in a state where the tilt exists in a first area among a plurality of areas obtained by dividing a space into the plurality of areas with a center point of the tilt as a center, the stop of tilt change executed immediately after the stop of tilt change executed in a state where the tilt exists in the first area is the stop of tilt change executed in a state where the tilt exists in a second area among the plurality of areas, and
the second area exists on a side opposite to the first area with respect to the center point.

4. The head tilt motion assistance system according to claim 2, wherein
the score determination criteria include a criterion for increasing the score in a case where a stop of change in the tilt in the head tilt motion is executed in all a state where the tilt exists in a first area among a plurality of areas obtained by dividing a space into the plurality of areas with a center point of the tilt as a center, a state where the tilt exists in a second area among the plurality of areas, a state where the tilt exists in a third area among the plurality of areas, and a state where the tilt exists in a fourth area among the plurality of areas,
the second area exists on a side opposite to the first area with respect to the center point,
the third area exists with 90° shift from the second area around the center point, and
the fourth area exists on a side opposite to the third area with respect to the center point.

5. The head tilt motion assistance system according to claim 2, wherein
the score determination criteria include a criterion according to a duration of a stop of change in the tilt in the head tilt motion,
the motion assistance notification unit determines the score on a basis of at least the score determination criteria and an actual duration of the stop of change in the tilt in the head tilt motion,
a range consisting of all of time that the duration of the stop of change in the tilt in the head tilt motion can be is divided into a plurality of levels, and
the criterion according to the duration of the stop of change in the tilt in the head tilt motion is a criterion in which higher score corresponds to a level associated with a longer duration of the stop of change in the tilt in the head tilt motion.

6. The head tilt motion assistance system according to claim 2, wherein
the score determination criteria include a criterion according to a relaxation degree of the user in the head tilt motion, and
the motion assistance notification unit determines the score on a basis of at least the score determination criteria and an actual relaxation degree of the user in the head tilt motion,
a range consisting of all of degree that the relaxation degree of the user in the head tilt motion can be is divided into a plurality of levels, and
the criterion according to the relaxation degree of the user in the head tilt motion is a criterion in which higher score corresponds to a level associated with a higher relaxation degree of the user in the head tilt motion.

7. The head tilt motion assistance system according to claim 2, wherein
the score determination criteria include a criterion according to an execution detail of preprocessing of the head tilt motion,
the motion assistance notification unit determines the score on a basis of at least the score determination criteria and an actual execution detail of the preprocessing of the head tilt motion,
the criterion according to the execution detail of preprocessing of the head tilt motion includes a criterion according to a degree of execution of the preprocessing,
a range consisting of all of degree that the degree of the execution of the preprocessing can be is divided into a plurality of levels, and
the criterion according to the degree of the execution of the preprocessing is a criterion in which higher score corresponds to a level associated with a higher degree of the execution of the preprocessing.

8. The head tilt motion assistance system according to claim 2, wherein
the score determination criteria include a criterion according to an execution detail of preprocessing of the head tilt motion,
the motion assistance notification unit determines the score on a basis of at least the score determination criteria and an actual execution detail of the preprocessing of the head tilt motion,
the criterion according to the execution detail of preprocessing of the head tilt motion includes a criterion according to a quantity of a type of an executed preprocessing,
a range consisting of all of quantity that the quantity of the type of the executed preprocessing can be is divided into a plurality of levels, and
the criterion according to the quantity of the type of the executed preprocessing is a criterion in which higher score corresponds to a level associated with a larger quantity of the type of the executed preprocessing.

9. The head tilt motion assistance system according to claim 2, wherein
the motion assistance notification includes a notification of a reason for the score.

10. The head tilt motion assistance system according to claim 2, wherein
the motion assistance notification unit sets the score determination criteria for a case where a fatigue degree of a brain of the user after execution of the head tilt motion is greater than or equal to a specific degree such that the score is determined to be low as compared with the score determination criteria for a case where the fatigue degree of the brain of the user after execution of the head tilt motion is less than the specific degree, and
the fatigue degree of the brain of the user is acquired by at least one of input by the user and measurement by a test.

11. The head tilt motion assistance system according to claim 1, wherein
the motion assistance notification includes a notification of a state of a stop of change in the tilt, and
the motion assistance notification unit makes the notification of the state of the stop of change in the tilt during execution of the head tilt motion.

12. The head tilt motion assistance system according to claim 1, wherein
the motion assistance notification includes a notification of a relaxation degree of the user, and
the motion assistance notification unit makes the notification of the relaxation degree of the user during execution of the head tilt motion.

13. The head tilt motion assistance system according to claim 1, wherein
the motion assistance notification includes a notification of an instruction to execute preprocessing of the head tilt motion, and
the motion assistance notification unit makes the notification of the instruction to execute the preprocessing of the head tilt motion before executing of the head tilt motion.

14. The head tilt motion assistance system according to claim 1, wherein
the motion assistance notification includes a notification of the tilt that is a recommended tilt,
the motion assistance notification unit makes the notification of the recommended tilt during execution of the head tilt motion,
the motion assistance notification unit identifies an actual execution detail of the head tilt motion on a basis of the tilt detected by the head tilt detection unit,
the motion assistance notification unit determines the recommended tilt on a basis of at least the actual execution detail of the head tilt motion and recommended tilt determination criteria that are criterions for determination of the recommended tilt, and
the recommended tilt determination criteria include a criterion according to an execution detail of the head tilt motion.

15. The head tilt motion assistance system according to claim 14, wherein
the recommended tilt determination criteria include a criterion for determining the tilt in a second area among a plurality of areas obtained by dividing a space into the plurality of areas with a center point of the tilt as a center as the recommended tilt when, in a case where a stop of tilt change that is a stop of change in the tilt in the head tilt motion is executed in a state where the tilt exists in the first area among the plurality of areas, the stop of tilt change executed immediately before the stop of tilt change executed in a state where the tilt exists in the first area is not the stop of tilt change executed in a state where the tilt exists in the second area among the plurality of areas, and
the second area exists on a side opposite to the first area with respect to the center point.

16. The head tilt motion assistance system according to claim 15, wherein
the recommended tilt determination criteria include a criterion for determining the tilt in a third area among the plurality of areas as the recommended tilt when, in a case where the stop of tilt change is executed in a state where the tilt exists in the first area, the stop of tilt change executed immediately before the stop of tilt change executed in a state where the tilt exists in the first area is the stop of tilt change executed in a state where the tilt exists in the second area, and
the third area exists with 90° shift from the second area around the center point.

17. The head tilt motion assistance system according to claim 1, further comprising a γ wave light output unit that outputs γ wave light that is light blinking at a frequency identical to a frequency of a γ wave of brain waves, wherein
the motion assistance notification unit causes the γ wave light output unit to output the γ wave light during execution of the head tilt motion.

18. The head tilt motion assistance system according to claim 1, further comprising a γ wave sound output unit that outputs γ wave sound identical in frequency to a γ wave of brain waves, wherein
the motion assistance notification unit causes the γ wave sound output unit to output the γ wave sound during execution of the head tilt motion.

19. The head tilt motion assistance system according to claim 1, wherein
the motion assistance notification unit executes a test for measuring a fatigue degree of a brain of the user after execution of the head tilt motion, and
the motion assistance notification includes a notification of the fatigue degree of the brain measured by the test.

20. The head tilt motion assistance system according to claim 1, wherein
the motion assistance notification includes a notification of an image representing a state where cerebrospinal fluid moves in a brain in a gravity direction, and
the motion assistance notification unit identifies a gravity direction with respect to the head on a basis of the tilt detected by the head tilt detection unit in the head tilt motion, and changes the gravity direction with respect to the brain in the image according to the gravity direction identified.

21. A non-transitory computer-readable storage medium storing a head tilt motion assistance program causing a computer to implement a motion assistance notification unit that makes a motion assistance notification that is a notification for assisting in a head tilt motion that is a motion in which a user tilts a head of the user, wherein
the motion assistance notification unit makes the motion assistance notification on a basis of the tilt detected by a head tilt detection unit that detects the tilt of the head.
